(19) United States
(12) Patent Application Publication
(10) Pub. No.: US 2018/0154142 A1
(43) Pub. Date: Jun. 7, 2018

(54) METHODS AND DEVICES FOR TREATMENT OF TUMORS WITH NANO-PULSE STIMULATION

(71) Applicant: Old Dominion University Research Foundation, Norfolk, VA (US)

(72) Inventors: Siqi Guo, Virginia Beach, VA (US); Stephen J. Beebe, Norfolk, VA (US); Richard Heller, Norfolk, VA (US)

(73) Assignee: Old Dominion University Research Foundation, Norfolk, VA (US)

(21) Appl. No.: 15/831,845

(22) Filed: Dec. 5, 2017

Related U.S. Application Data
(60) Provisional application No. 62/434,574, filed on Dec. 15, 2016.
(60) Provisional application No. 62/430,214, filed on Dec. 5, 2016.

(51) Int. Cl. A61N 1/36; A61K 45/00; A61N 1/05; A61P 35/00

(52) U.S. Cl. CPC A61N 1/36002; A61P 35/00; A61N 1/05; A61K 45/00

(57) ABSTRACT

Disclosed herein are methods and devices for stimulating an immune response to a disease in a subject, which involves passing sub-microsecond long pulses of electric fields having an amplitude between 5 kV/cm and 68 kV/cm through an abnormal growth of a subject sufficient to suppress myeloid-derived suppressor cell (MDSC) or regulatory T cell (Treg) production, increase adenosine triphosphate (ATP) or high mobility group box 1 (HMGB1) production, or stimulate dendritic cell activation in the subject.

### CROSS-REFERENCES TO RELATED APPLICATIONS

This application claims the benefit of U.S. Provisional Application No. 62/434,574, filed Dec. 15, 2016, and U.S. Provisional Application No. 62/430,214, filed Dec. 5, 2016, the contents of which are hereby incorporated by reference in their entireties for all purposes.

### FIELD OF INVENTION

The methods disclosed herein relate generally to the use of sub-microsecond electrical pulses for tumor ablation. More specifically, the methods disclosed herein describe the use of sub-microsecond electrical pulses at a localized tumor site in order to induce anti-tumor immunity and reduce distant metastases.

### BACKGROUND

Ablation refers to a wide variety of minimally invasive surgical methods used to treat and remove tumors and other abnormal growth, for example, cancerous tumors. Ablation often involves heating, vaporization, chipping, or other erosive processes that are used to slowly remove the tumor in a controlled manner. For example, special probes or electrodes are often used (e.g., to emit radio frequency waves) in order to “burn” and remove the tumors.

An emerging modality for tumor ablation is electroporation using ultra-short, high-field strength electric pulses, also frequently referred to as nano-pulse stimulation (NPS). In this approach, a generator is used to create high amplitude electric pulses of very short duration (e.g., less than 1 microsecond) that are applied to the tumor using electrodes. The electric pulses induce a voltage across the cell membranes of the tumor cells that leads to opening of pores in the cell membranes, either temporarily or permanently. Using the pulses to open pores in the cell membrane of the tumor cells may ultimately trigger apoptosis (i.e. programmed cell death) and the death of the tumor cells.

Thus, NPS tumor ablation is an approach that is distinguishable from conventional local tumor (e.g., cancer) therapy, surgery, chemotherapy or radiation therapy. For example, in reference to cancers, it shifts chemistry-based cancer therapy to a physical cancer treatment modality, since it does not involve delivery of any DNA or virus, checkpoint inhibitors, engineered patient immune cells, or a molecule of any kind. Furthermore, NPS tumor ablation is also distinguishable from other forms of ablation in that it is a minimally invasive, ionizing radiation-free, and non-thermal technique for local tumor elimination when appropriately applied.

As a physical treatment modality, the direct effects of nano-pulse stimulation of local cancer tumors, such as apoptosis, have been studied extensively. However, there remains much unknown about the direct and indirect effects of nano-pulse stimulation at the local tumor and far away from the ablation site (e.g., at distant tumors).

### BRIEF SUMMARY

According to one aspect, a method is disclosed for stimulating an immune response to a disease in a subject, the method including passing sub-microsecond long pulses of electric fields having an amplitude between 5 kV/cm and 68 kV/cm through an abnormal growth of a subject sufficient to suppress myeloid-derived suppressor cell (MDSC) production in the subject, wherein at least a 35% decrease in the MDSC concentration confirms immune response stimulation.

In some embodiments, a method is disclosed for stimulating an immune response to a disease in a subject, the method including passing sub-microsecond long pulses of electric fields through an abnormal growth of a subject sufficient to suppress myeloid-derived suppressor cell (MDSC) production in the subject, the electric fields having an amplitude between 5 kV/cm and 68 kV/cm, ordering, directing or performing a comparison of a pre-treatment measurement and a post-treatment measurement of an MDSC concentration in the subject, and verifying that the treatment of pulsed electric fields sufficiently suppressed MDSC production based on the comparison, a greater than 35% decrease in the MDSC concentration confirming immune response stimulation.

In some embodiments, the method further includes ordering, directing or performing a comparison of a pre-treatment measurement and a post-treatment measurement of an MDSC concentration in the subject, the pre-treatment measurement occurring, for example, between 0 and 2 days before the treatment, the post-treatment measurement occurring, for example, between 2 and 7 days after the treatment. In some embodiments, the method further includes ordering, directing or performing a calculation of a percentage change in the MDSC concentration based on the pre-treatment measurement and the post-treatment measurement. In some embodiments, the method further includes ordering, directing, or directly performing a collection of a pre-treatment measurement, a post-treatment measurement, or both. In some embodiments, the pre-treatment and post-treatment measurements are from blood samples of the subject or biopsies of the abnormal growth. In some embodiments, the method further includes introducing an epigenetic modulator or a phosphoinositide 3-kinase (PI3K) inhibitor into a microenvironment of the abnormal growth.

In some embodiments, the epigenetic modulator includes one or more of the following: 5-azacytidine, 5-aza-20-deoxycytidine, zebularine, epigallocatechin-3-gallate, suberanilohydroxamic acid (vorinostat), romidepsin, entinostat, trichostatin A (TSA), sodium butyrate, or valproic acid (VPA). In some embodiments, the PI3K inhibitor includes one or more of the following: wortmannin, demethoxyviridin, LY294002, idelalisib, perifosine, buparlisib, duvelisib, alpelisib, TGR 1202, copanlisib, PX-866, dactolisib, RP6530, SF1126, INK1117, pictilisib, XL147, XL765, palomid 529, GSK1059615, ZSTK474, PWT33597, CUDC-907, ME-401, IPI-549, IC87114, TG100-115, CAL263, RP6503, PI-103, GNE-477, or AEZS-136.

In some embodiments, the abnormal growth is a breast cancer tumor. In some embodiments, the subject is a human. In some embodiments, the passing step includes passing the pulses at a frequency between 0.5 Hz and 7 Hz and each of the pulses is between 7 ns to 300 ns in duration.

In some embodiments, the method includes suppressing production of a regulatory T cell (Treg) in the subject, stimulating release of adenosine triphosphate (ATP) from the abnormal growth, and stimulating a release of high mobility group box 1 (HMGB1) from the abnormal growth, wherein at least a 35% respective increase or decrease of any one or more of the Treg, ATP, or HMGB1 confirms immune response stimulation.

According to another aspect, a method is disclosed for stimulating an immune response to a disease in a subject, the method including passing sub-microsecond long pulses of electric fields having an amplitude between 5 kV/cm and 68 kV/cm through an abnormal growth of a subject sufficient to suppress regulatory T cell (Treg) production in the subject, wherein at least a 35% decrease in the Treg concentration confirms immune response stimulation.

In some embodiments, the method may further include ordering, directing or performing a comparison of a pre-treatment measurement and a post-treatment measurement of the Treg concentration in the subject, the pre-treatment measurement occurring between 0 and 2 days before the pulses, the post-treatment measurement occurring between 2 and 7 days after the pulses. The method may also include verifying that the treatment of pulsed electric fields sufficiently suppressed Treg production. In some embodiments, the method may further include ordering, directing or performing a calculation of a percentage change in the Treg concentration based on the pre-treatment measurement and the post-treatment measurement. In some embodiments, the method may further include applying a second treatment of sub-microsecond long pulses of electric fields through the abnormal growth of a subject based on the comparison. In some embodiments, the method may further include ordering, directing, or directly performing a collection of a pre-treatment measurement, a post-treatment measurement, or both. In some embodiments, the pre-treatment and post-treatment measurements are from blood samples of the subject or biopsies of the abnormal growth. In some embodiments, the method further includes introducing an epigenetic modulator or a phosphoinositide 3-kinase (PI3K) inhibitor into a microenvironment of the abnormal growth.

In some embodiments, the epigenetic modulator includes one or more of the following: 5-azacytidine, 5-aza-20-deoxycytidine, zebularine, epigallocatechin-3-gallate, suberanilohydroxamic acid (vorinostat), romidepsin, entinostat, trichostatin A (TSA), sodium butyrate, or valproic acid (VPA). In some embodiments, the PI3K inhibitor includes one or more of the following: wortmannin, demethoxyviridin, LY294002, idelalisib, perifosine, buparlisib, duvelisib, alpelisib, TGR 1202, copanlisib, PX-866, dactolisib, RP6530, SF1126, INK1117, pictilisib, XL147, XL765, palomid 529, GSK1059615, ZSTK474, PWT33597, CUDC-907, ME-401, IPI-549, IC87114, TG100-115, CAL263, RP6503, PI-103, GNE-477, or AEZS-136.

In some embodiments, the abnormal growth is a breast cancer tumor. In some embodiments, the subject is a human. In some embodiments, the passing step includes passing the pulses at a frequency between 0.5 Hz and 7 Hz and each of the pulses is between 7 ns to 300 ns in duration.

In some embodiments, the method includes suppressing production of a myeloid-derived suppressor cell (MDSC) in the subject, stimulating release of adenosine triphosphate (ATP) from the abnormal growth, and stimulating a release of high mobility group box 1 (HMGB1) from the abnormal growth, wherein at least a 35% respective increase or decrease of any one or more of the MDSC, ATP, or HMGB1 confirms immune response stimulation.

According to a further aspect, a method is disclosed for stimulating an immune response to a disease in a subject, the method including passing sub-microsecond long pulses of electric fields having an amplitude between 5 kV/cm and 68 kV/cm through an abnormal growth of a subject sufficient to stimulate release of adenosine triphosphate (ATP) in the subject, wherein at least a 35% increase in ATP concentration confirms immune response stimulation.

In some embodiments, the method further includes ordering, directing or performing a comparison of a pre-treatment measurement and a post-treatment measurement of the ATP concentration in the subject, the pre-treatment measurement occurring between 0 hours and 48 hours before the pulses, the post-treatment measurement occurring between 4 hours and 24 hours after the pulses. The method may also comprise verifying that the treatment of pulsed electric fields sufficiently stimulated ATP release. In some embodiments, the method further includes ordering, directing or performing a calculation of a percentage change in the ATP concentration based on the pre-treatment measurement and the post-treatment measurement. In some embodiments, the method further includes applying a second treatment of sub-microsecond long pulses of electric fields through the abnormal growth of a subject based on the comparison. In some embodiments, the method further ordering, directing, or directly performing a collection of a pre-treatment measurement, a post-treatment measurement, or both. In some embodiments, the pre-treatment and post-treatment measurements are from blood samples of the subject or biopsies of the abnormal growth. In some embodiments, the method further includes introducing an epigenetic modulator or a phosphoinositide 3-kinase (PI3K) inhibitor into a microenvironment of the abnormal growth.

In some embodiments, the epigenetic modulator includes one or more of the following: 5-azacytidine, 5-aza-20-deoxycytidine, zebularine, epigallocatechin-3-gallate, suberanilohydroxamic acid (vorinostat), romidepsin, entinostat, trichostatin A (TSA), sodium butyrate, or valproic acid (VPA). In some embodiments, the PI3K inhibitor includes one or more of the following: wortmannin, demethoxyviridin, LY294002, idelalisib, perifosine, buparlisib, duvelisib, alpelisib, TGR 1202, copanlisib, PX-866, dactolisib, RP6530, SF1126, INK1117, pictilisib, XL147, XL765, palomid 529, GSK1059615, ZSTK474, PWT33597, CUDC-907, ME-401, IPI-549, IC87114, TG100-115, CAL263, RP6503, PI-103, GNE-477, or AEZS-136.

In some embodiments, the abnormal growth is a breast cancer tumor. In some embodiments, the subject is a human. In some embodiments, the passing step includes passing the pulses at a frequency between 0.5 Hz and 7 Hz and each of the pulses is between 7 ns to 300 ns in duration.

In some embodiments, the method includes suppressing production of a myeloid-derived suppressor cell (MDSC) in the subject, suppressing production of a regulatory T cell (Treg) in the subject, and stimulating a release of high mobility group box 1 (HMGB1) from the abnormal growth, wherein at least a 35% respective increase or decrease of any one or more of an MDSC, Treg, or HMGB1 concentration confirms immune response stimulation.

According to yet another aspect, a method is disclosed herein for stimulating an immune response to a disease in a subject, the method comprising: passing sub-microsecond long pulses of electric fields having an amplitude between 5 kV/cm and 68 kV/cm through an abnormal growth of a subject sufficient to stimulate release of high mobility group box 1 (HMGB1) in the subject, wherein at least a 35% increase in HMGB1 concentration confirms immune response stimulation.

In some embodiments, the method may further include ordering, directing or performing a comparison of a pre-treatment measurement and a post-treatment measurement of the HMGB1 concentration in the subject, the pre-treatment measurement occurring between 0 hours and 48 hours before the pulses, the post-treatment measurement occurring between 4 hours and 24 hours after the pulses. The method may also comprise verifying that the treatment of pulsed electric fields sufficiently stimulated HMGB1 release. In some embodiments, the method further includes ordering, directing or performing a calculation of a percentage change in the HMGB1 concentration based on the pre-treatment measurement and the post-treatment measurement. In some embodiments, the method further includes applying a second treatment of sub-microsecond long pulses of electric fields through the abnormal growth of a subject based on the comparison. In some embodiments, the method further includes ordering, directing, or directly performing a collection of a pre-treatment measurement, a post-treatment measurement, or both. In some embodiments, the pre-treatment and post-treatment measurements are from blood samples of the subject or biopsies of the abnormal growth. In some embodiments, the method further includes introducing an epigenetic modulator or a phosphoinositide 3-kinase (PI3K) inhibitor into a microenvironment of the abnormal growth.

In some embodiments, the epigenetic modulator includes one or more of the following: 5-azacytidine, 5-aza-20-deoxycytidine, zebularine, epigallocatechin-3-gallate, suberanilohydroxamic acid (vorinostat), romidepsin, entinostat, trichostatin A (TSA), sodium butyrate, or valproic acid (VPA). In some embodiments, the PI3K inhibitor includes one or more of the following: wortmannin, demethoxyviridin, LY294002, idelalisib, perifosine, buparlisib, duvelisib, alpelisib, TGR 1202, copanlisib, PX-866, dactolisib, RP6530, SF1126, INK1117, pictilisib, XL147, XL765, palomid 529, GSK1059615, ZSTK474, PWT33597, CUDC-907, ME-401, IPI-549, IC87114, TG100-115, CAL263, RP6503, PI-103, GNE-477, or AEZS-136.

In some embodiments, the abnormal growth is a breast cancer tumor. In some embodiments, the subject is a human. In some embodiments, the passing step includes passing the pulses at a frequency between 0.5 Hz and 7 Hz and each of the pulses is between 7 ns to 300 ns in duration.

In some embodiments, the method includes suppressing production of a myeloid-derived suppressor cell (MDSC) in the subject, suppressing production of a regulatory T cell (Treg) in the subject, and stimulating release of adenosine triphosphate (ATP) from the abnormal growth, wherein at least a 35% respective increase or decrease of any one or more of a Treg, MDSC, or ATP concentration confirms immune response stimulation.

According to a further aspect, a method is disclosed herein for stimulating an immune response to a disease in a subject, the method including passing sub-microsecond long pulses of electric fields through an abnormal growth of a subject sufficient to stimulate dendritic cell activation in the subject, the electric fields having an amplitude between 5 kV/cm and 68 kV/cm, ordering, directing, or performing a comparison of a pre-treatment measurement and a post-treatment measurement of a cluster of differentiation 40 (CD40) concentration, a cluster of differentiation 80 (CD80) concentration, a cluster of differentiation 86 (CD86) concentration, or a major histocompatibility complex class II (MHC-II) molecule concentration in the subject, the pre-treatment measurement occurring within 48 hours before the treatment, the post-treatment measurement occurring between 24 hours and 48 hours after the treatment, and verifying that the treatment of pulsed electric fields sufficiently stimulated dendritic cell activation based on the comparing, at least 35% increase in the CD40, CD80, CD86, or MHC-II molecule concentration confirming immune response stimulation.

According to another aspect, a method is disclosed herein for stimulating an immune response to a disease in a subject, the method including stimulating dendritic cell activation in a subject by passing sub-microsecond long pulses of electric fields through an abnormal growth of the subject, the electric fields having an amplitude between 5 kV/cm and 68 kV/cm, wherein at least a 35% increase in concentration of one or more of the following: 1) a cluster of differentiation 40 (CD40), 2) a cluster of differentiation 80 (CD80), 3) a cluster of differentiation 86 (CD86), or 4) a major histocompatibility complex class II (MHC-II) molecule confirms immune response stimulation.

In some embodiments, the method further includes calculating a percentage change in the CD40, CD80, CD86, or MHC-II molecule concentration based on a pre-treatment measurement and a post-treatment measurement, the pre-treatment measurement occurring between 0 hours and 48 hours before the pulses, the post-treatment measurement occurring between 24 hours and 48 hours after the pulses. In some embodiments, the method further includes ordering, directing, or directly performing a collection of the pre-treatment measurement, the post-treatment measurement, or both. In some embodiments, the pre-treatment and post-treatment measurements are from biopsies of the abnormal growth or sentinel lymph nodes of the subject. In some embodiments, the abnormal growth is a breast cancer tumor. In some embodiments, the abnormal growth is a malignant tumor, and the method further comprises preventing or at least reducing metastases distant to the malignant tumor.

According to additional aspect, devices configured to perform some or all of the steps of various methods of the present disclosure are provided. For example, in some embodiments a device comprises a generator configured to generate sub-microsecond long pulses of electric fields having an amplitude between 5 kV/cm and 68 kV/cm, the sub-microsecond long pulses of electric fields sufficient to suppress either one or both of myeloid-derived suppressor cell (MDSC) and regulatory T cell (Treg) production in a subject when applied to an abnormal growth of the subject; and an electrode configured to apply the sub-microsecond long pulses of electric field to the abnormal growth of the subject.

In some embodiments, the device may comprise a pre-treatment collector configured to collect pre-treatment blood of the subject prior to applying the sub-microsecond long pulses of electric fields to the abnormal growth of the subject and a post-treatment collector configured to collect post-treatment blood of the subject after applying the sub-microsecond long pulses of electric field to the abnormal growth of the subject. The device may also comprise a processor in electronic communication with computer-readable memory, the computer-readable memory storing instructions that, when executed by the processor, cause the processor to: calculate a pre-treatment measurement of a myeloid-derived suppressor cell (MDSC) concentration and/or a regulatory T cell (Treg) concentration in the pre-treatment blood; calculate a post-treatment measurement of the MDSC concentration and/or Treg concentration in the post-treatment blood; and compare the pre-treatment measurement and the post-treatment measurement. In some embodiments the results of the comparison may be displayed or otherwise indicated through a user interface.

In some embodiments, a device comprises a generator configured to generate sub-microsecond long pulses of electric fields having an amplitude between 5 kV/cm and 68 kV/cm, the sub-microsecond long pulses of electric field sufficient to stimulate release in a subject of adenosine triphosphate (ATP) and/or high mobility group box 1 (HMGB1) when applied to an abnormal growth of the subject; and an electrode configured to apply the sub-microsecond long pulses of electric field to the abnormal growth of the subject. The device may also comprise a pre-treatment collector configured to collect pre-treatment blood of the subject prior to applying the sub-microsecond long pulses of electric fields to the abnormal growth of the subject and a post-treatment collector configured to collect post-treatment blood of the subject after applying the sub-microsecond long pulses of electric field to the abnormal growth of the subject. The device may also comprise a processor in electronic communication with computer-readable memory, the computer-readable memory storing instructions that, when executed by the processor, cause the processor to: calculate a pre-treatment measurement of ATP and/or HMGB1 in the pre-treatment blood; calculate a post-treatment measurement of ATP and HMGB1 in the post-treatment blood; and compare the pre-treatment measurement and the post-treatment measurement. In some embodiments the results of the comparison may be displayed or otherwise indicated through a user interface.

In some embodiments a device comprises a generator configured to stimulate dendritic cell activation in a subject by passing sub-microsecond long pulses of electric field through an abnormal growth of the subject sufficient to increase concentration of one or more of the following: 1) a cluster of differentiation 40 (CD40), 2) a cluster of differentiation 80 (CD80), 3) a cluster of differentiation 86 (CD86), or 4) a major histocompatibility complex class II (MHC-II) molecule. The device may also comprise a processor configured to perform one or more steps of the various methods described in the present disclosure.

In certain implementations, the devices according to the present disclosure may include one or more processors configured to execute machine-readable instructions; a memory for storing machine-readable instructions; and wherein the one or more processors are connected to the memory to execute the machine-readable instructions comprising the steps for implementing the methodologies described herein. The device may also include an input/output interface connected to the one or more processors to allow a user to interact with the device, for example, the input/output interface may include a display.

Other features and advantages of the devices and methodology of the present disclosure will become apparent from the following detailed description of one or more implementations when read in view of the accompanying figures. Neither this summary nor the following detailed description purports to define the invention. The invention is defined by the claims.

### BRIEF DESCRIPTION OF THE DRAWINGS

FIG. 1 illustrates schematically an example of a system for generating and delivering nanosecond electrical pulses, in accordance with embodiments of the present disclosure.

FIG. 2 illustrates an example of a nanosecond pulse generator, in accordance with embodiments of the present disclosure.

FIG. 3 illustrates a perspective view of an example of an applicator tip with electrodes, that may be utilized in various embodiments of the present disclosure.

FIG. 4 illustrates a perspective view of an example electrode, in accordance with embodiments of the present disclosure.

FIG. 5 is a flow chart depicting an example of a method of electrical pulse treatment for suppression of Myeloid-derived suppressor cell (MDCSC) and regulatory T cell (Treg) production, in accordance with embodiments of the present disclosure.

FIG. 6A is a plot of breast cancer tumor size in mice following either no treatment or a single treatment of administering sub-microsecond electrical pulses, in accordance with embodiments of the present disclosure.

FIG. 6B is a plot of tumor luminescence in mice following either no treatment or a single treatment of administering sub-microsecond electrical pulses, in accordance with embodiments of the present disclosure.

FIG. 6C is a plot of tumor luminescence in mice following a single treatment of administering sub-microsecond electrical pulses, in accordance with embodiments of the present disclosure.

FIG. 6D is a plot of tumor luminescence in mice following a single treatment of administering sub-microsecond electrical pulses, in accordance with embodiments of the present disclosure.

FIG. 7A illustrates tumor luminescence in a control group of mice at day 6 following inoculation, in accordance with embodiments of the present disclosure.

FIG. 7B illustrates tumor luminescence in a group of mice at day 6 following inoculation and treated with 300 pulses, in accordance with embodiments of the present disclosure.

FIG. 7C illustrates tumor luminescence in a group of mice at day 6 following inoculation and treated with 1000 pulses, in accordance with embodiments of the present disclosure.

FIG. 7D illustrates tumor luminescence in a control group of mice at day 17 following inoculation, in accordance with embodiments of the present disclosure.

FIG. 7E illustrates tumor luminescence in a group of mice at day 17 following inoculation and treated with 300 pulses, in accordance with embodiments of the present disclosure.

FIG. 7F illustrates tumor luminescence in a group of mice at day 17 following inoculation and treated with 1000 pulses, in accordance with embodiments of the present disclosure.

FIG. 8A is a plot of metastases size in a control group of mice over time, in accordance with embodiments of the present disclosure.

FIG. 8B is a plot of metastases size in mice over time following the treatment of a local tumor with sub-microsecond duration electrical pulses, in accordance with embodiments of the present disclosure.

FIG. 8C is Kaplan-Meier survival curves for two groups of mice treated with or without electrical pulses, in accordance with embodiments of the present disclosure.

FIG. 9A illustrates in vivo imaging for luciferase expression indicating organ metastases in a group of mice without electrical pulse treatment, in accordance with embodiments of the present disclosure.

FIG. 9B illustrates in vivo imaging for luciferase expression indicating organ metastases in a group of mice without electrical pulse treatment, in accordance with embodiments of the present disclosure.

FIG. 9C illustrates in vivo imaging for luciferase expression indicating organ metastases in a group of mice treated with electrical pulses, in accordance with embodiments of the present disclosure.

FIG. 9D illustrates in vivo imaging for luciferase expression indicating organ metastases in a group of mice treated with electrical pulses, in accordance with embodiments of the present disclosure.

FIG. 10A is a table of quantified data for organ metastases following the treatment of a local tumor with or without sub-microsecond duration electrical pulses, in accordance with embodiments of the present disclosure.

FIG. 10B is a bar graph of photons received from organ metastases through in vitro imaging following the treatment of a local tumor with or without sub-microsecond duration electrical pulses, in accordance with embodiments of the present disclosure.

FIG. 11A illustrates in vivo imaging after secondary tumor challenge for a group of tumor-free mice, in accordance with embodiments of the present disclosure.

FIG. 11B illustrates in vivo imaging for a group of control mice injected with tumors, in accordance with embodiments of the present disclosure.

FIG. 12A is a plot of photons received after secondary tumor challenge for mice treated with or without sub-microsecond duration electrical pulses, in accordance with embodiments of the present disclosure.

FIG. 12B is table quantifying the results of secondary tumor challenge for mice treated with or without sub-microsecond duration electrical pulses, in accordance with embodiments of the present disclosure.

FIG. 13A is a bar graph illustrating levels of CD4+ memory T cells in various groups of mice, in accordance with embodiments of the present disclosure.

FIG. 13B is a bar graph illustrating levels of CD8+ memory T cells in various groups of mice, in accordance with embodiments of the present disclosure.

FIG. 13C illustrates flow cytometry data for IFN-γ producing CD4+ T cells in a control group of mice, in accordance with embodiments of the present disclosure.

FIG. 13D illustrates flow cytometry data for IFN-γ producing CD4+ T cells in a tumor group of mice, in accordance with embodiments of the present disclosure.

FIG. 13E illustrates flow cytometry data for IFN-γ producing CD4+ T cells in a NPS-treated, tumor-free group of mice, in accordance with embodiments of the present disclosure.

FIG. 13F illustrates flow cytometry data for IFN-γ producing CD8+ T cells in a control group of mice, in accordance with embodiments of the present disclosure.

FIG. 13G illustrates flow cytometry data for IFN-γ producing CD8+ T cells in a tumor group of mice, in accordance with embodiments of the present disclosure.

FIG. 13H illustrates flow cytometry data for IFN-γ producing CD8+ T cells in a NPS-treated tumor-free group of mice, in accordance with embodiments of the present disclosure.

FIG. 14A is a bar graph illustrating the amounts of IFN-γ producing CD4+ T cells for three groups of mice, in accordance with embodiments of the present disclosure.

FIG. 14B is a bar graph illustrating the amounts of IFN-γ producing CD8+ T cells for three groups of mice, in accordance with embodiments of the present disclosure.

FIG. 14C is a bar graph illustrating the amounts of IFN-γ product from splenocytes for three groups of mice after 24 h incubation with tumor lysate, in accordance with embodiments of the present disclosure.

FIG. 15A illustrates flow cytometry data associated with regulator T cells (Tregs) in a control group of mice, in accordance with embodiments of the present disclosure.

FIG. 15B illustrates flow cytometry data associated with regulator T cells (Tregs) in a tumor group of mice, in accordance with embodiments of the present disclosure.

FIG. 15C illustrates flow cytometry data associated with myeloid derived suppressor cells (MDSCs) in the blood of a control group of mice, in accordance with embodiments of the present disclosure.

FIG. 15D illustrates flow cytometry data associated with myeloid derived suppressor cells (MDSCs) in the blood of a tumor group of mice, in accordance with embodiments of the present disclosure.

FIG. 15E illustrates flow cytometry data associated with regulator T cells (Tregs) in a tumor-free group of mice, in accordance with embodiments of the present disclosure.

FIG. 15F illustrates flow cytometry data associated with myeloid derived suppressor cells (MDSCs) in the blood of a tumor-free group of mice, in accordance with embodiments of the present disclosure.

FIG. 15G illustrates flow cytometry data associated with myeloid derived suppressor cells (MDSCs) in the blood of a group of tumor regressing mice, in accordance with embodiments of the present disclosure.

FIG. 15H illustrates flow cytometry data associated with myeloid derived suppressor cells (MDSCs) in the blood of a group of tumor growing mice, in accordance with embodiments of the present disclosure.

FIG. 16A is a bar graph of summary data associated with the reversal of Treg levels after electrical pulse treatment, in accordance with embodiments of the present disclosure.

FIG. 16B is a bar graph of summary data associated with the reversal of MDSC levels after electrical pulse treatment, in accordance with embodiments of the present disclosure.

FIG. 17A is flow cytometer data of the amount of CD11B collected from a control group of mice, in accordance with embodiments of the present disclosure.

FIG. 17B is flow cytometer data of the amount of CD11C collected from a control group of mice, in accordance with embodiments of the present disclosure.

FIG. 17C illustrates levels of CD40 in a control group of mice, in accordance with embodiments of the present disclosure.

FIG. 17D is flow cytometer data of the amount of FOXP3 collected from a control group of mice, in accordance with embodiments of the present disclosure.

FIG. 17E is flow cytometer data of the amount of CD11B collected from a treatment group of mice, in accordance with embodiments of the present disclosure.

FIG. 17F is flow cytometer data of the amount of CD11C collected from a treatment group of mice, in accordance with embodiments of the present disclosure.

FIG. 17G illustrates levels of CD40 in a treatment group of mice, in accordance with embodiments of the present disclosure.

FIG. 17H is flow cytometer data of the amount of FOXP3 collected from a treatment group of mice, in accordance with embodiments of the present disclosure.

FIG. 17I is a bar graph of the amount of MDSCs in two groups of mice, in accordance with embodiments of the present disclosure.

FIG. 17J is a bar graph of the amount of DCs in two groups of mice, in accordance with embodiments of the present disclosure.

FIG. 17K is a bar graph of the amount of CD40+ DCs in two groups of mice, in accordance with embodiments of the present disclosure.

FIG. 17L is a bar graph of the amount of Tregs in two groups of mice, in accordance with embodiments of the present disclosure.

FIG. 18A is a bar graph of levels of calreticulin expression associated with various groups of dendritic cells, in accordance with embodiments of the present disclosure.

FIG. 18B is a bar graph of levels of adenosine triphosphate (ATP) expression associated with various groups of dendritic cells, in accordance with embodiments of the present disclosure.

FIG. 18C is a bar graph of levels of High mobility group box 1 (HMGB1) protein expression associated with various groups of dendritic cells, in accordance with embodiments of the present disclosure.

FIG. 18D is a bar graph of levels of co-stimulatory molecule (IA/IE, CD40, and CD86) expression for three types of dendritic cells, in accordance with embodiments of the present disclosure.

FIG. 19 illustrates the dose response of sub-microsecond duration electrical pulses for 4T1-luc cells, in accordance with embodiments of the present disclosure.

FIG. 20A plots the growth curve of a tumor without electrical pulse treatment, in accordance with embodiments of the present disclosure.

FIG. 20B plots the growth curve of a tumor with NPS, in accordance with embodiments of the present disclosure.

FIG. 21A plots the growth curve of tumors with or without electrical pulse treatment, showing two distinctive growth patterns of tumors following electrical pulse treatment, in accordance with embodiments of the present disclosure.

FIG. 21B is a table summarizing the growth rates shown in FIG. 21A, in accordance with embodiments of the present disclosure.

FIG. 22A illustrates splenomegaly in 4T1-luc tumor burden mice, in accordance with embodiments of the present disclosure.

FIG. 22B illustrates the spleen of control mice, in accordance with embodiments of the present disclosure.

FIG. 22C is a bar chart comparing the spleen weight of 4T1-luc tumor burden mice to control mice, in accordance with embodiments of the present disclosure.

FIG. 22D is a bar chart comparing white blood cell counts in the blood and spleen of 4T1-luc tumor burden mice to control mice, in accordance with embodiments of the present disclosure.

FIG. 23A is flow cytometry data for Th17 cells in blood of control mice, in accordance with embodiments of the present disclosure.

FIG. 23B is flow cytometry data for Th17 cells in blood of tumor mice, in accordance with embodiments of the present disclosure.

FIG. 23C is flow cytometry data for Th17 cells in blood of tumor-free mice, in accordance with embodiments of the present disclosure.

FIG. 23D is a bar chart illustrating the significant increase of Th17 cells in the blood of tumors and a reversal to normal levels after electrical pulse treatment, in accordance with embodiments of the present disclosure.

FIG. 24 is a bar chart illustrates dendritic cell activation by electrical pulse treatment, in accordance with embodiments of the present disclosure.

### DETAILED DESCRIPTION

In the following detailed description, reference is made to the accompanying drawings that show, by way of illustration, some examples of embodiments in which the disclosure may be practiced. With reference to the above-listed drawings, this section describes particular embodiments and their detailed construction and operation. The embodiments described herein are set forth by way of illustration only and not limitation. Those skilled in the art will recognize in light of the teachings herein that, for example, other embodiments are possible, variations can be made to the example embodiments described herein, and there may be equivalents to the components, parts, or steps that make up the described embodiments.

Terms.

A “tumor” includes any neoplasm or abnormal, unwanted growth of tissue on or within a subject, or as otherwise known in the art. A tumor can include a collection of one or more cells exhibiting abnormal growth. There are many types of tumors. A malignant tumor is cancerous, a pre-malignant tumor is precancerous, and a benign tumor is noncancerous. Thus, an abnormal, uncontrolled growth of tissue, may include those that are cancerous, precancerous, and benign. Examples of tumors include a benign prostatic hyperplasia (BPH), breast cancer tumors, uterine fibroid, pancreatic carcinoma, liver carcinoma, kidney carcinoma, colon carcinoma, pre-basal cell carcinoma, and tissue associated with Barrett's esophagus. Other examples of tumors or abnormal growth include adipose tissue or fat, warts, calluses, corns, skin lesions, and other types of unwanted cosmetic/dermal growths.

A “nanosecond electric pulse” or a “sub-microsecond electric pulse”, sometimes abbreviated as nsEP, refers to an electrical pulse with a length or width of between 0.1 nanoseconds (ns) to 1000 nanoseconds, or as otherwise known in the art. A plurality of nanosecond electric pulses may be used to generate a nanosecond pulsed electric field.

A “nanosecond pulsed electric field”, sometimes abbreviated as nsPEF, includes an electric field of nanosecond electric pulses having a pulse width of between 0.1 nanoseconds (ns) to 1000 nanoseconds, or as otherwise known in the art. It is sometimes referred to as sub-microsecond pulsed electric field. NsPEFs often have high peak voltages, such as 5 kilovolts per centimeter (kV/cm), 10 kV/cm, 20 kV/cm, to 500 kV/cm. NsPEFs have been found to trigger both necrosis and apoptosis in cancerous tumors. The application of nsPEFs is sometimes referred to as nano-pulse stimulation (NPS), which can involve the selective treatment of tumors with nsPEFs to induce apoptosis within the tumor cells without substantially affecting normal cells in the surrounding tissue due to its non-thermal nature. Treatment of biological cells with nsPEF often uses a multitude of periodic pulses at a frequency ranging from 0.1 per second (Hz) to 10,000 Hz.

Illustrative embodiments are now discussed. Other embodiments may be used in addition or instead. Details which may be apparent or unnecessary may be omitted to save space or for a more effective presentation. Conversely, some embodiments may be practiced without all of the details which are disclosed. Some details and features described in reference to one embodiment may be used with other embodiments.

There have been extensive studies on the outcomes associated with electroporation that directly results from applying nano-pulse stimulation to a local cancer tumor. Nanosecond length electric pulses or nanosecond pulsed electric fields (nsPEFs) with short rise and fall times and high electric field strength, when applied to mammalian cells, can have the direct effect of permeabilizing both plasma and organelle membranes (e.g., electroporation), which allows entry of disruptive small ions, disturbs intracellular vesicles, and releases calcium from endoplasmic reticulum stores. Previous studies have shown that electrical pulses may directly induce cell death in the treated tumor via mechanisms such as caspase-dependent or independent apoptosis, necrosis, and necrosis defined as parthanatos.

However, nano-pulse stimulation may actually trigger mechanisms that result in longer-lasting, farther-reaching results that go beyond merely killing tumor cells via ablation. As an example of one such consequence described herein, the application of nano-pulse stimulation with certain parameters to the tumor of a subject may actually have immunogenic effects that can provide a vaccine-like, anti-tumor immunity. For instance, it will be shown herein that for mice inoculated with poorly immunogenic, metastatic 4T1-luc mouse mammary carcinoma (similar to late stage breast cancer in humans), a single application of nanosecond electrical pulses can result in not only complete regression of the tumor in some cases, but also the prevention of spontaneous distant organ metastases—even in mice that exhibit incomplete tumor regression. For the mice that do have complete tumor regression, the mice are protected from secondary tumor cell challenge.

This vaccine-like anti-tumor immunity is likely the result of the electrical pulses destroying the tumor microenvironment (and reducing its accompanying immune suppressor cells) and inducing drastic increases in long-term memory T cells, which are vital to the immune response. Furthermore, the tumor cells treated with electrical fields exhibit release of danger associated molecular patterns (DAMPs), including calreticulin, HMGB1 and ATP, and also activate dendritic cells. Such findings suggest that electrical pulse stimulation is a potent immunogenic cell death inducer to elicit anti-tumor immunity, in addition to its already-known use for local tumor eradication based on apoptosis or necrosis.

This allows nanosecond electric pulse ablation to be used not only, for example, to treat local cancer tumors (e.g., early-stage cancer), but also extends its use as a novel, minimally invasive immunotherapeutic strategy to treat advanced-stage cancers where the cancer has metastasized and multiple tumors are present. In such applications, treatment with electrical pulses having certain parameters discussed herein can be applied at a local tumor site in order to induce antitumor immunity and prevent distant metastases.

In order to generate these electrical pulses to be used for a treatment, a special device should be used. In particular, special generators are best to consistently produce nanosecond length electric pulses or nanosecond pulsed electric fields (nsPEFs) with short rise and fall times and high electric field strength to treat cancer. Furthermore, the electrical pulses may cause varying effects on the cells, such as stimulation, suppression, damage, or even death (e.g., via apoptosis or necrosis). The exact effect may depend on the parameters of the electric field (e.g., pulse strength, frequency, and number of pulses) and the cell types or status. Accordingly, the device used to generate the electrical pulses should be configurable and able to generate electrical pulses of varying parameters in order to cause the desired outcome.

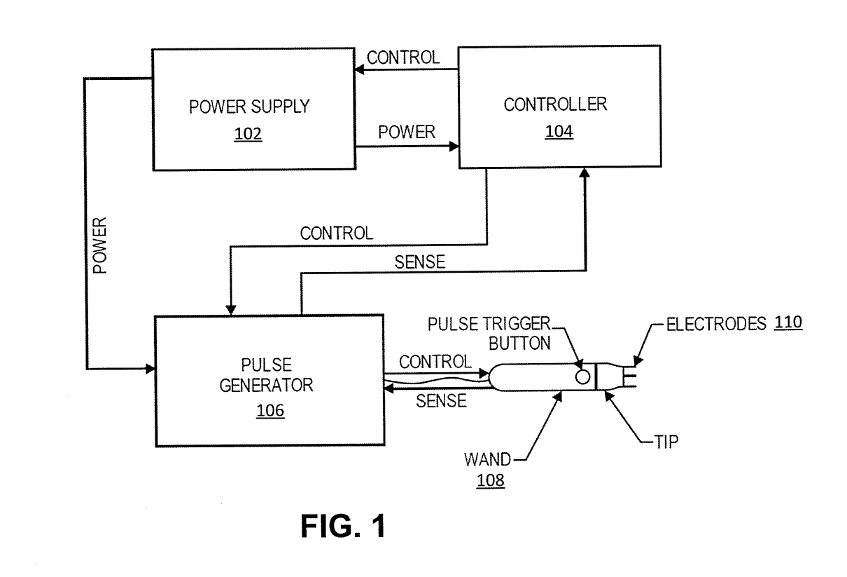

FIG. 1 illustrates an example of such a system for generating and delivering electrical sub-microsecond (e.g., nanosecond) duration pulses.

In some embodiments, the system may include a power supply 102, a controller 104, a pulse generator 106, and a wand 108 having one or more electrodes 110. The power supply 102 may supply power to the controller 104 and the pulse generator 106.

The pulse generator 106 may generate electrical pulses that are conducted by the electrodes 110 of the wand 108. The electrodes 110 of the wand 108 may be applied to tissue of a subject in order to pulse an electric field through the tissue.

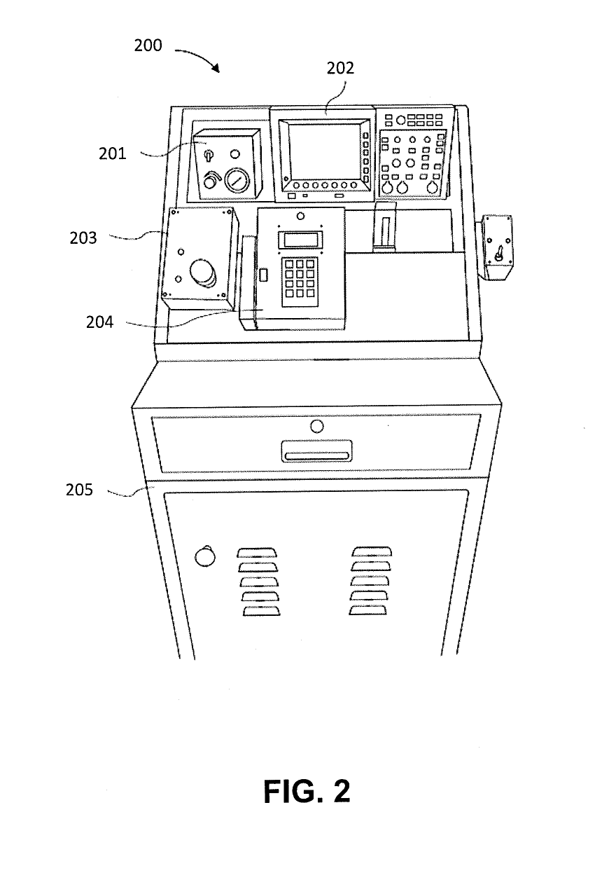

FIG. 2 illustrates an example of a nanosecond pulse generator, in accordance with embodiments of the present disclosure. The nanosecond pulse generator 200, which represents an example of an embodiment of the pulse generator 106 described in regards to FIG. 1, may generate electrical pulses of sub-microsecond duration with the electrical pulses having variably configurable parameters. For instance, the nanosecond pulse generator 200 is configured such that it may be capable of changing pulse widths, duty cycles, and other pulse parameters for the generated electrical pulses. In some embodiments, pulse widths, duty cycles, and other pulse parameters are controlled by a spark gap, the critical distance of which is controlled by compressed gas, such as compressed carbon dioxide. In other embodiments, the electrical pulse parameters are controlled by solid state devices.

In some embodiments, the nanosecond pulse generator 200 may include pressure readout 201, digitizing oscilloscope 202, emergency off button 203, and microcontroller interface 204. These components may all be connected (directly or indirectly through one or more intervening components) to the nanosecond pulse generator 200 within a metal-shielded cabinet 205.

A human operator may input a number of pulses, amplitude, and frequency, for example, using a touch screen, stylus, pen, keyboard, a numeric keypad of the microcontroller interface 204, or any other input device. In some embodiments, the pulse width is fixed. A microcontroller of the nanosecond pulse generator 200 sends signals to a high voltage power supply (HVPS) and pressure system to control a spark gap (switch) within the cabinet 205. Fiber optic cables electrically isolate the contents of the metal cabinet with the nanosecond pulse generator 200 from the outside. In order to further isolate the generator, the nanosecond pulse generator 200 may be battery powered instead of from a wall outlet.

Other examples of high voltage pulse generators, besides the nanosecond pulse generator 200 shown in the figure, that may be configured to be used with different embodiments and methods of the present disclosure, can be seen in: Gundersen et al. “Nanosecond Pulse Generator Using a Fast Recovery Diode”, IEEE 26.sup.th Power Modulator Conference, 2004, pages 603-606; Tang et al. “Solid-State High Voltage Nanosecond Pulse Generator,” IEEE Pulsed Power Conference, 2005, pages 1199-1202; Tang et al. “Diode Opening Switch Based Nanosecond High Voltage Pulse Generators for Biological and Medical Applications”, IEEE Transactions on Dielectrics and Electrical Insulation, Vol. 14, No. 4, 2007, pages 878-883; Yampolsky et al., “Repetitive Power Pulse Generator With Fast Rising Pulse” U.S. Pat. No. 6,831,377; Schoenbach et al. “Method and Apparatus for Intracellular Electro-Manipulation”, U.S. Pat. No. 6,326,177; Gundersen et al., “Method for Intracellular Modifications Within Living Cells Using Pulsed Electric Fields”, U.S. Patent Application No. 2006/0062074; Kuthi et al., “High Voltage Nanosecond Pulse Generator Using Fast Recovery Diodes for Cell Electro-Manipulation”, U.S. Pat. No. 7,767,433; Krishnaswamy et al., “Compact Subnanosecond High Voltage Pulse Generation System for Cell Electro-Manipulation”, U.S. Patent Application No. 2008/0231337; and Sanders et al. “Nanosecond Pulse Generator”, U.S. Patent Application No. 2010/0038971. The entire content of these publications is incorporated herein by reference.

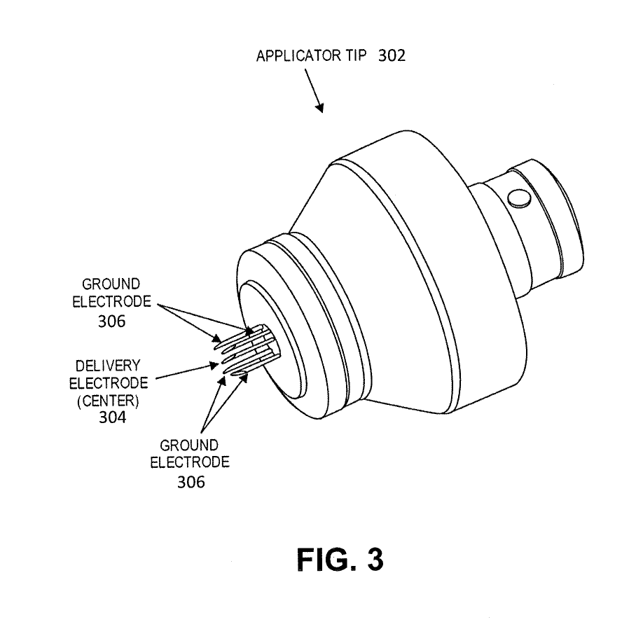

FIG. 3 illustrates a perspective view of an example of an applicator tip with electrodes, which may be used in various embodiments of the present disclosure. In particular, an applicator tip 302 is shown that has one delivery electrode 304 and four ground electrodes 306 surrounding the delivery electrode 304.

The nanosecond electrical pulses produced by a generator can be delivered to tissue by using applicator tip 302 (e.g., on a wand, such as the wand 108 shown in FIG. 1). In the exemplary embodiments, each electrode was constructed by using a 30 gauge needle (i.e. about 0.255 mm in diameter). The delivery electrode 304 and the ground electrodes 306 have the same length for each applicator tip 302.

In other embodiments, this length may vary in the range of about 2 millimeters (mm) to 5 mm. The electrodes may be placed to form a square pattern, with the ground electrodes 306 at the corners of this square and the delivery electrode 304 at its center. Center-to-center distance between the delivery electrode 304 and each of the ground electrodes 306 is about 1.75 mm. This configuration provides a volume of about 30.625 cubic-millimeters (mm³) within the boundary formed by the ground electrodes 306. The ground electrodes 306 and the delivery electrode 304 are electrically isolated from each other by embedding them in a Teflon® insulation.

The tip configuration may be different than illustrated. There may be other applicator tip configurations suitable for applying electrical pulses to tissue. These configurations may include tips comprising at least one delivery electrode and at least one ground electrode. For example, as the system disclosed above is coaxial in nature, with the ground electrodes surrounding the delivery electrode, any number of needle configurations may be realized, including a circular arrangement with five or more ground electrodes, a triangular arrangement with three ground electrodes, wherein the delivery electrode may be placed at the geometrical center of such arrangements. A simple linear arrangement with just two opposing electrodes, i.e., one return electrode and one delivery electrode, may also be used for the delivery of the electrical pulses.

Still other tip configurations, for example those with different electrode spacing or length, may also be used for the delivery of electrical pulses to tissue. However, as the effect of these short pulses on cells is largely dependent upon the strength of electric field, an increase in return and active electrode spacing may have to be accompanied by a proportional increase in output voltage to maintain the required field for the effect on cells. Similarly, if the spacing is reduced, the voltage could be proportionally decreased.

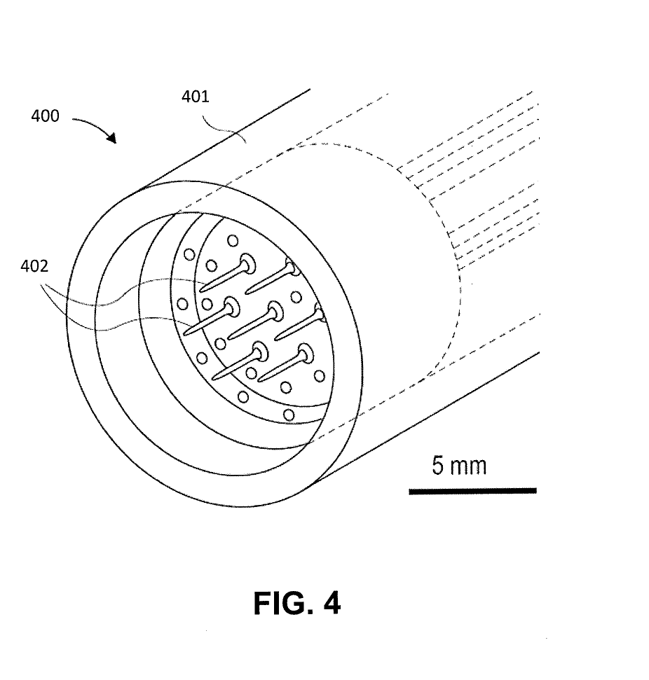

FIG. 4 illustrates a perspective view of an example electrode, in accordance with embodiments of the present disclosure. In particular, the figure shows a seven-needle suction electrode 400. In electrode 400, the sheath 401 surrounds seven sharp needle electrodes 402 with an opening at a distal end. When the open, distal end is placed against tissue, air is evacuated from the resulting chamber sufficient to draw tissue (e.g., the entire tumor or a portion thereof) into the chamber. The tumor is drawn so that one or more of the needle electrodes 402 preferably penetrates the tumor. In this respect, the needle electrodes 402 are configured to pierce the tumor. The center needle is at one polarity, and the outer six needles are at the opposite polarity. An electric field can then be precisely applied to the tumor using the electrode 400 to conduct the electric pulses produced by the generator, such as generator 200 shown in FIG. 2.

The needle electrodes 402 can be apposed, one of each positive and negative pair of electrodes on one side of a tumor and the other electrode of the pair on an opposing side of the tumor. Opposing sides of a tumor can include areas outside or within a tumor, such as if a needle electrode 402 pierces a portion of the tumor.

It should be noted that this is only an example configuration of the electrode. The nature of the electrode used mainly depends upon the shape of the tumor or other abnormal growth. Its physical size and stiffness can also be taken into account in selection of a particular electrode type.

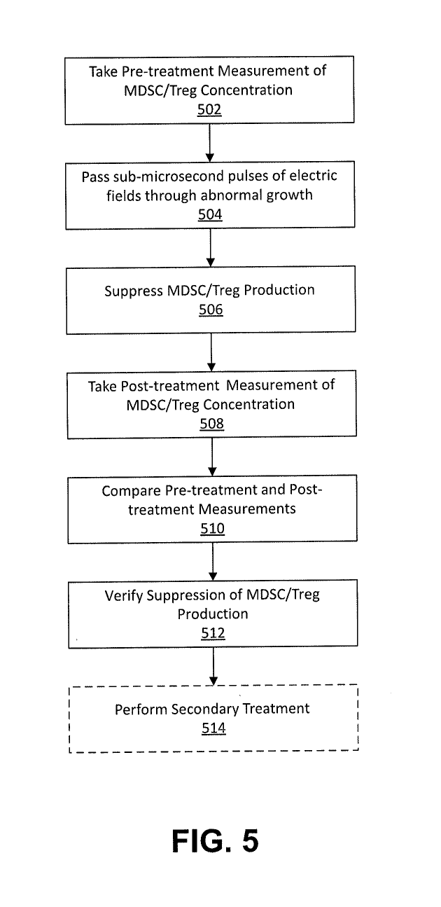

The following figures, FIGS. 5-24, are associated with novel observations attained from studies involving treatment of tumors in mice with sub-microsecond duration electric pulses of certain parameters (e.g., after they are produced by the generator and applied to the tumor via electrode). For context, these studies are associated with a particular interest in the interaction of sub-microsecond duration electric pulses with certain types (e.g., metastatic) of breast cancer, however, it should be understood that methodology of the present disclosure is not limited to treatment of breast cancer.

Breast cancer is the second leading cause of morbidity and mortality due to cancer in women. Metastatic mammary cancer presents considerable therapeutic challenges due to disease heterogeneity, absence of established therapeutic targets and poor prognosis. Despite great advances in therapeutic strategies, metastatic breast cancer has no preferred treatments, and there are no available cures. Surgery, hormonal therapy, radiation and cytotoxic chemotherapy are the likely treatment options. Commonly used chemotherapeutic agents like anthracyclines, taxanes and cytophosphanes not only present significant toxicities, but also promote aggressive resistant types of breast cancer that include high numbers of mammary cancer stem cells.

Although a number of therapies are available for breast cancer patients, advanced stages, especially metastatic diseases, are typically associated with serious complications, deterioration of life quality, a worse prognosis, morbidities and mortalities. Novel effective therapeutic strategies for metastatic eradication/control are an unmet urgent medical need for patient survival and an improved quality of life.

Thus, in order to address these issues and seriously evaluate the potential of NPS ablation as an effective therapeutic strategy against such metastatic diseases, studies (from which FIGS. 5-24 are based) were undertaken to determine if NPS ablation could effectively ablate primary cancer and induce antitumor immunity to help control distant metastasis. These studies were conducted in mice that were inoculated with poorly immunogenic, metastatic 4T1-luc mouse mammary carcinoma. This poorly immunogenic and metastatic 4T1 mouse orthotopic tumor model has well-established definitions of metastatic breast cancer and is one of the few breast cancer models that exhibits the capability to metastasize to the lungs, liver, brain, and bones, closely mimicking advanced-stage breast cancer in humans. Accordingly, the results observed in these studies should be similar (or even predictive) to the results of electrical pulse treatment of metastatic cancers in humans.

For the materials used in the studies, the specific cell lines include 4T1 cells (CRL2539™ ATCC, MD, USA) maintained in high glucose RPMI-1640 (ATCC® 30-2001™) supplemented with 10% FBS (Atlantic Biological, FL, USA), 100 IU of penicillin and 100 μg/ml streptomycin. The 4T1-luc cells were purchased from Dr. G Gary Sahagian at Tufts University and were maintained in high glucose DMEM (ATCC® 30-2002™) supplemented 10% FBS, NEAA and antibiotics (100 units/ml penicillin and 100 μg/ml streptomycin).

The reagents and antibodies used included D-Luciferin purchased from Goldbio Technology (St Louis, Mo.). WST-1 for cell viability assay was obtained from Roche Applied Science (Indianapolis, Ind.). Collagenase I, DNase I (D4527, Sigma), Monensin solution (1000×), True True-Nuclear™ Transcription Factor Buffer Set and antibodies, anti-mouse CD16/32, LEAF™ Purified antimouse CD3, Pacific Blue™ anti-mouse CD3, FITC anti-mouse CD4, PerCP anti-mouse CD8a, PE anti-mouse/human CD11b, APC/Cy7 anti-mouse Ly-6G/Ly-6C (Gr-1), PE/Cy7 anti-mouse CD62L, APC anti-mouse/human CD44, APC anti-mouse CD25, Biolegend PE antimouse/rat/human FOXP3, PerCP anti-mouse CD11c, PE/Cy7 anti-mouse CD86, FITC antimouse I-A/I-E, APC anti-mouse CD40, Pacific Blue™ anti-mouse F4/80, PE anti-mouse IFN-γ, and PE/Cy7 anti-mouse IL-17A were purchased from Biolegend (San Diego, Calif.). HSP 70 Antibody (C92F3A-5) and ATP Determination Kit were ordered from (Thermo-Fisher). Mouse High Mobility Group box 1 Protein (HMGB1) ELISA kit was purchased from MyBioSource. Anticalreticulin antibody (Alexa Fluor® 488) (ab196158) was obtained from Abcam.

The mice used were Female Balb/c mice (8-10 weeks of age) purchased from Jackson Laboratory (Bar Harbor, Me.) or Harlan Laboratories (Dublin, Va.), housed and maintained in the ODU AAALAC approved animal facility.

In the studies, the mice were injected with 1×106 4T1-luc or 4T1 cells in 50 μL Dulbecco's phosphate buffered saline with calcium and magnesium (DPBS) in the posterior part of the mammary fat pad. The mice were weighed before and post-treatment (e.g., with electrical pulses) and twice weekly. Mice were euthanized at the end of the follow-up period or at specified time points described in experimental designs or when they meet the criteria described at experimental endpoints in the approved IACUC protocol. In vivo treatment with electrical pulses was performed by delivering electrical pulses to tumor tissue using a five needle electrode array, two-needle or a suction two-plate electrode both with a 5 mm gap, such as described above. There is a 5 mm gap between a central anode and four cathode pins for a five needle electrode. The high voltage pulses were delivered to the center needle. Care was taken with to ensure the needles are only within the the tumor mass during treatment. The varying pulse parameters were pulse duration 100 ns, frequency 1 to 2 Hz, applied electric fields 50 KV (with a range of 46-54 KV/cm) and pulse number 300, 600, or 1000 depending on experimental designs. To prevent breakdown over the skin and burning, it was necessary to thoroughly remove all hair from the treatment area by shaving closely and using a depilatory cream such as Nair to remove all hair shafts in the treatment area. It is also important that the electrodes are covered with an insulating fluid, such as K-Y jelly or ultrasound gel, such that electrodes are not exposed to air.

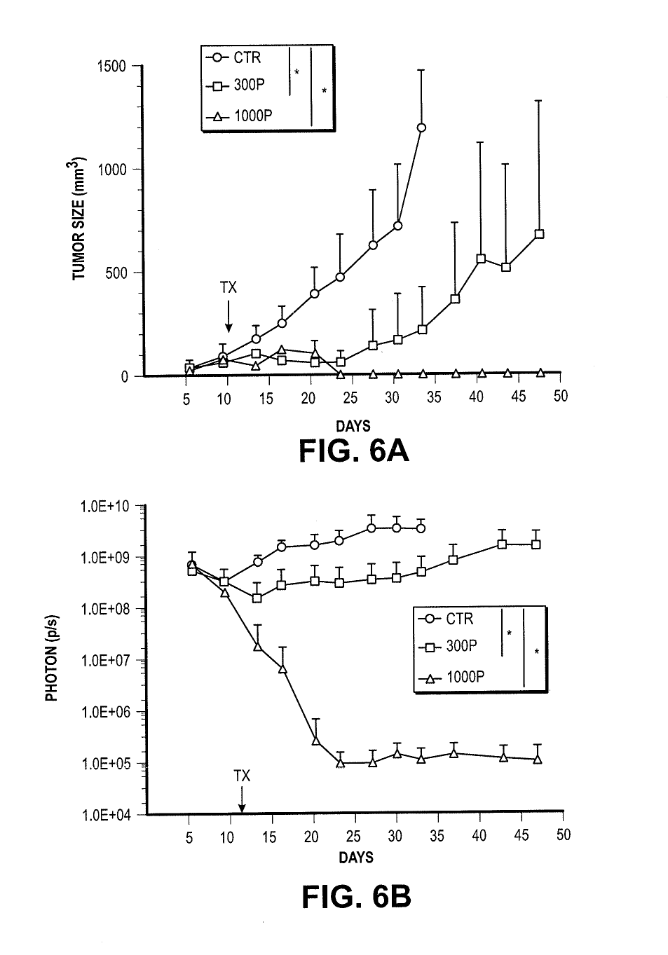

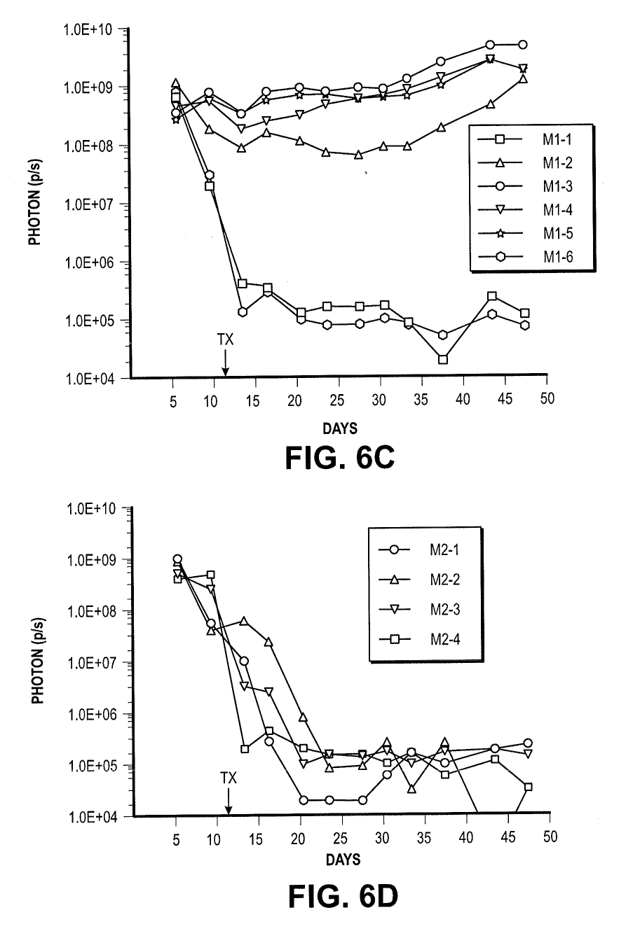

Standardized procedures were used to generate the data used in the graphs and reports of FIGS. 6A-24, which include tumor size, cell viability, actual images of tumors and organs, the concentration or percentage of specific cells or particulates in the tumor site or blood, and so forth. For example, the size of primary tumors in the mice were assessed by digital calipers and the Xenogen IVIS Spectrum system (Caliper Life Sciences, Hopkinton, Mass.). Tumor volume (e.g., as an indicator of tumor size, reported in FIGS. 6A-6D, 8A-8C, 20A-20B, and 21A-21B) was determined using the following formula: V=πab2/6, where (a) is the longest diameter and (b) is the shortest diameter perpendicular to (a). The mice were monitored for tumor growth or regression for the duration of the experiment or until primary tumor volumes reached 1500 mm³.

Since luciferase expression in 4T1-luc cells is stable during in vitro culture, total luciferase expression was measured and calculated to ascertain cell viability (e.g., the mortality of tumor cells following treatment). In this case, after 18 hour incubation, total luciferase expression of control cells without NPS treatment was seen as 100% cell viable, and viability of cells treated with NPS was determined by a formula: Viability of treated cells=Luciferase expression of treated cells/Luciferase expression of control cells×100%.

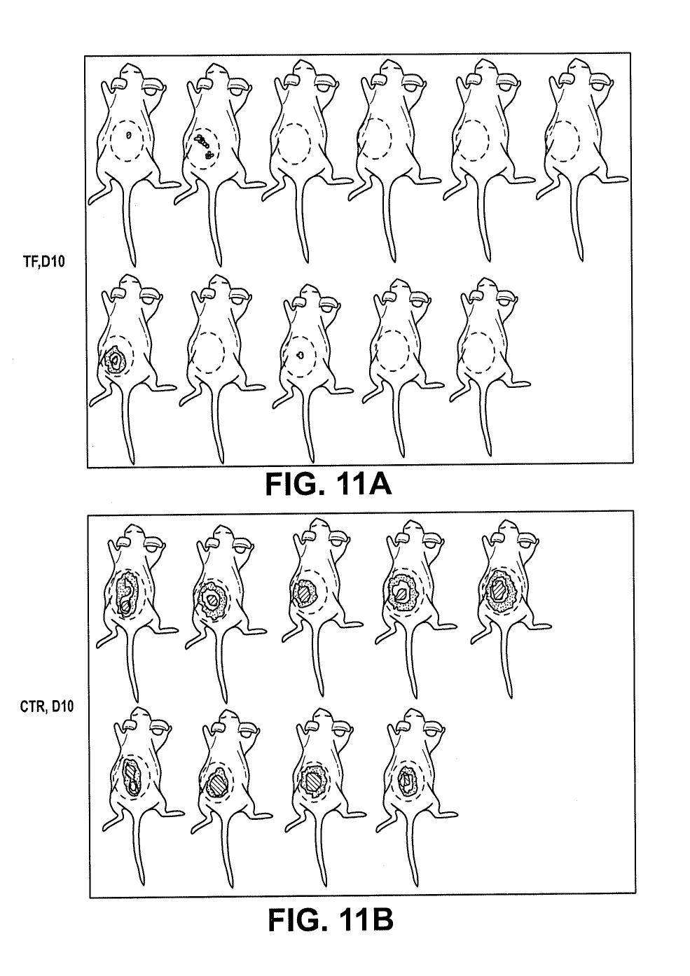

To perform in vivo or ex vivo imaging (e.g., such as in FIGS. 11A-11B), at various times or twice a week, the mice were administrated intraperitoneally with D-luciferin 150 μg/gram body weight in PBS (stock solution 15 mg/ml). After 10 to 15 min, the mice were imaged under anesthesia with 2.5% isofluorane in a Xenogen IVIS 100 spectrum. At experimental endpoints, luciferin-injected mice were euthanized and organs were removed and imaged within 30 minutes. Luminescence is expressed as photons/sec/ROI (region of interest) minus background luminescence for a similarly sized region.

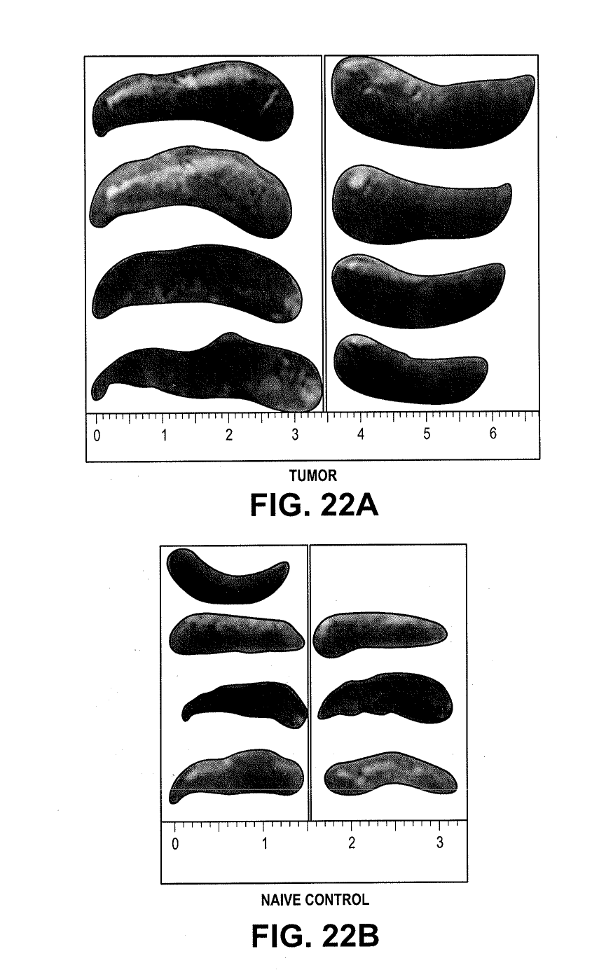

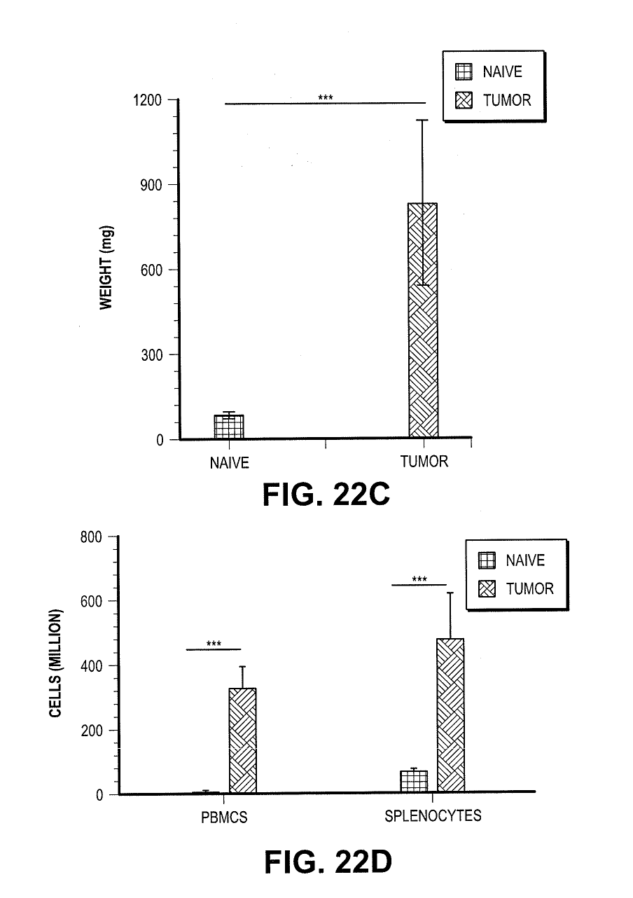

To isolate peripheral blood monocytes (PBMCs), splenocytes, and tumor infiltrate cells (e.g., as reported in graph 2206 of FIG. 22), blood from the mice was collected by tail vein bleeding or cardiac puncture. After blood was spun down, supernatant was removed then mixed with 10×ACK lysis buffer (1 ml/0.1 ml volume of blood) at room temperature in the dark for 10 min. Cells were washed with DPBS twice and prepared for culture or staining. Spleens were cut into pieces and minced in 40-μm nylon cell strainers. As mentioned above, lysis of red blood cells was mixed with ACK lysis buffer, 2-10 ml depending on the size of spleens. Tumors were minced into thin pieces and were dissociated in collagenase I (200 U/ml), DNase I (200 U/mL; Sigma-Aldrich), in complete culture medium for 1 h at 37° C. Dissociated tumor tissues were then passed through a 40-μm nylon cell strainer to obtain a single-cell suspension.

To perform Ex vivo splenocyte re-stimulation with anti-CD3 Ab or tumor lysate, one day prior to incubation, low endotoxin/azide free LEAF anti-CD3 Ab (0.5 μg/ml in DPBS) was coated into a 24-well plate and incubated at 4° C. Anti-CD3 coated wells were washed with DPBS three times before cells were added. Splenocytes (2 million/ml) 1 ml per well were incubated with media, tumor lysate (10 μg/ml) or plate bound anti-CD3 Ab in a 24-well plate. For intracellular cytokine staining, cells were incubated for 6 hours and monensin added for the final 4 hours. For IFN-γ production, cells were incubated for 24 hours and supernatants were collected for ELISA assay.

To determine cell surface calreticulin (CRT) and release of adenosine triphosphate (ATP) and High mobility group box 1 (HMGB1) protein from NPS treated breast cancer cells (e.g., the release of DAMPs associated with treatment), 0.1 ml 4T1 cells (5×106/ml) in complete media were loaded in a 1 mm gap cuvette and treated with NPS with 60 ns, 50 kV/cm, 1 Hz and 60 pulses which was determined by viability assay with >90% cell death. Cells then were placed in a 6 well plate for 1 hour or 20 hours. Cells with media but no NPS treatment served as negative control and a known ICD inducer, mitoxantrone (1 μM) was added to cells as a positive control. Cell surface-exposed CRT was measured by flow cytometry (FACSAria). Extracellular ATP concentrations were quantified by the luciferase/luciferin-based ATP determination Kit (A22066, Thermo-Fisher). The amount of HMGB1 in the supernatant was determined by HMGB1 ELISA Kit (MBS722248, MyBioSource).

To prepare and stimulate bone marrow-derived dendritic cells (BMDCs), BMDCs were prepared from harvested bone marrow cells by 8 day incubation in the presence of 20 ng/ml GM-CSF (R&D). BMDCs were stimulated with media as a control, LPS (5 μg/ml) or CPG 1668 (5 μM) as positive controls, NPS treated 4T1 cells or directly treated with low dose of NPS (5-10 pulses, 60 ns, 50 kV/cm and 1 Hz). Cells were incubated at 37° C. with 5% CO₂ for 24 to 48 hours then analyzed for cell surface activation markers by flow cytometry. The cell surface activation markers include major histocompatibility complex class II molecules (MHC-II), cluster of differentiation 40 (CD40) protein and cluster of differentiation 86 (CD86) protein.

To perform flow cytometry for cell surface staining, 1 to 2 million PBMCs, splenocytes or tumor infiltrate cells in 100 μL complete media or FACS buffer (2% FBS DPBS) were added with the antibody mixture and incubated at room temperature for 30 min. Cells then were washed with 2 ml FACS buffer twice and re-suspended in 0.5 ml FACS buffer with 2.5% paraformaltehyde (PFA) for flow cytometric analysis by FACSAria (BD Biosciences). For Intracellular staining, cells were prepared by pre-incubation with purified anti-CD16/32 (Fc block), followed by surface labeling of cells with anti-CD4 FITC, anti-CD8 PerCP or anti-CD25 APC, followed by intracellular staining using mAbs anti-IL-17A PE-Cy7 and anti-IFN-γ PE after fixation and permeabilization with fixation and permeabilization buffer. In case of PE antimouse/rat/human FOXP3 staining, True-Nuclear™ Transcription Factor Buffer Set was used for fixation and permeabilization. Samples were analyzed on a flow cytometer (FACSAria, BD Biosciences).

Finally, in order to perform the statistical analysis in order to generate the reports and graphs associated with FIGS. 6A-24, all values are reported as the mean±standard deviation (SD). Analysis of tumor volume and growth curve (Area Under Curve) was by One Way ANOVA. Animal survival was analyzed with Kaplan-Meier Survival Analysis (LogRank test). One Way ANOVA (3 or more groups) or 2-tailed Student's t-test (2 groups) was utilized to analyze the quantitative data, such as regulatory T cells (Tregs), Myeloid-derive suppressor cells (MDSCs), dendritic cells (DCs), dendritic cell activation markers (e.g., cluster of differentiation 40 protein, or CD40), Inteferon gamma (IFN-γ) positive CD4+ or CD8+ T cells, Immunogenic cell death (ICD) markers such as calreticulin (CRT) or 70 kilodalton heat shock proteins (Hsp70) or adenine triphosphate (ATP) or High mobility group box 1 (HMGB1) protein, CD4/CD8+ T cells, weight of spleen, etc. The rate of organ metastasis between control and NPS treated animals or the rate of resistance to second tumor challenge between control and tumor free mice was analyzed by Chi-Square test. Statistical significance is assumed at p<0.05. All statistical analysis including Kaplan-Meier Survival Analysis was completed using the SigmaPlot 12.0 (Aspire Software International).

From the results of the studies, which are described in further detail herein, it was concluded that an electrical pulse treatment can be used in a novel manner for inducing an immunogenic response and suppressing MDSC/Treg production (e.g., in the tumor microenvironment). Accordingly, sub-microsecond duration electrical pulses can be implemented in a method for treating metastatic diseases (such as advanced-stage breast cancer).

FIG. 5 is a flow chart depicting an example of a method of electrical pulse treatment for suppression of MDCSC/Treg production, in accordance with embodiments of the present disclosure. Although the operations in FIG. 5 are described in the context of MDSC/Treg amounts, they may be applied to other markers, including calreticulin, ATP, HMGB1, CD80, CD40, CD86, and/or MHC2.

At block 502, a pre-treatment measurement may be taken of myeloid-derived suppressor cells (MDSC) and/or regulatory T cell (Tregs) concentration. For instance, blood can be extracted from a subject or a biopsy taken from the tumor site. In the case of blood, the white blood cells can be separated out. In the case of a biopsy, tumor cells may be separated out. Different particles of interest can be labeled with biomarkers (e.g., fluorescent antibodies) and provided to a flow cytometer for counting purposes. This approach can be used in order to determine the MDSC/Treg concentration. Measurements may also be taken of calreticulin, ATP, HMGB1, CD80, CD40, CD86, and/or MHC2. In general, a reading of a blood sample may be obtained faster and more easily than a biopsy analysis (which may require sending the biopsy to a lab). When a tumor is present (e.g., pre-treatment), MDSCs/Tregs are biomarkers for the tumor, and there should be higher levels of MDSCs and Tregs in the blood or tumor microenvironment.

At block 504, sub-microsecond pulses of electric fields are applied to an abnormal growth (e.g., a primary tumor) of the subject. These pulses will generally have parameters sufficient for reducing the tumor microenvironment and activating dendritic cells.

At block 506, the treatment of the abnormal growth with the sub-microsecond pulses should begin to suppress MDSC and/or Treg production within the subject (e.g., by reducing the immunosuppressive tumor microenvironment). Thus, reductions in MDSC/Treg levels in the blood or tumor microenvironment should indicate that the tumor is regressing. Conversely, the treatments should increase calreticulin, ATP, HMGB1 (which are released from tumor cell death), as well as increase CD80, CD40, CD86, and/or MHC2 (which are markers for dendritic cell activation).

At block 508, the suppression of MDSC and/or Treg production may be confirmed, for example, by taking a post-treatment measurement of the MDSC/Treg concentration. This post-treatment measurement should be taken from the same source as in block 502. For instance, if a blood sample was taken pre-treatment, a blood sample should be taken post-treatment. In some embodiments, measurements may also be taken of calreticulin, ATP, and HMGB1. A measurement may be taken of all three markers, because the three markers have different targets, different receptors they are activating, and different mechanisms of activating dendritic cells and macrophages. For instance, ATP attracts dendritic cells into entering the tumor microenvironment. In some embodiments, measurements may also be taken of CD80, CD40, CD86, and/or MHC2.

A suitable time for taking this post-treatment measurement may depend on the timeframe needed to observe a meaningful decrease or increase in the biomarker of interest. For example, a post-treatment blood measurement may be taken 2 days following treatment, which is how long it takes for MDSC or Treg levels to decrease in the blood. In contrast, increases in ATP may manifest 12-24 hours after treatment, increases in HMGB1 may manifest 24 hours or more after treatment, and increases in calreticulin may manifest between 6-10 hours after treatment and go away by 24 hours. Thus, multiple post-treatment measurements may need to be taken in order to capture increases across multiple markers (as in the case of capturing increases in all 3 of ATP, HMGB1, and calreticulin.

At block 510, the pre-treatment and post-treatment measurements of MDSC/Treg concentration are compared, and at block 512 the suppression of MDSC/Treg is verified. The treatment should have suppressed MDSC/Treg production (e.g., at block 506), which means that the post-treatment measurements of MDSC/Treg should be meaningfully lower. In some embodiments, a meaningful decrease in MDSC/Treg may refer to a 35% or greater decrease in MDSC or Tregs by two days or more following treatment. If there is a big difference in the blood samples, an even bigger difference would be expected at the tumor site.

If comparing calreticulin, ATP, and/or HMGB1, those levels should be meaningfully higher following treatment to verify that dendritic cells have been activated. In some embodiments, a meaningful increase in calreticulin, ATP, or HMGB1 may refer to a 35% or more increase in calreticulin at 6-10 hours following treatment, a 35% or greater increase in ATP at 12-24 hours following treatment, or a 35% or greater increase in HMGB1 at 24 hours or more following treatment.

If comparing CD80, CD40, CD86, and/or MHC2, those levels should be higher following treatment to verify that dendritic cells have been activated. A meaningful increase in this instance may refer to a 20% or greater increase in CD80, CD40, CD86, or MHC2 at 24-48 hours following treatment.

A table is provided below for these biomarkers disclosing some possible measurement sites, pre and post treatment measurement timing, and what constitutes a meaningful increase or decrease of the biomarker:

| | Where | When to | When to | | |
|---|---|---|---|---|---|
| | to | measure | measure after- | % | |
| Marker | measure | before | broadest range | increase/decrease | Comments |
| MDSC | blood or | 2 to 0 | 2 to 7 days after | ≥35% decrease | Levels of MDSCs in blood |
| | tumor | days | NPS | | appear correlated to tumor |
| | | before | | | burdens or indicate the |
| | | NPS | | | success of Tx. |
| | | | | | Decrease of MDSCs in tumor |
| | | | | | may benefit immune |
| | | | | | outcome. |
| Treg | blood or | 2 to 0 | 2 to 7 days after | ≥35% decrease | Decrease of Tregs in tumor |
| | tumor | days | NPS | | presumably promotes immune |
| | | before | | | response. |
| | | NPS | | | |
| ATP | Tumor, | 48-0 | 4-24 hours after | ≥35% increase | ATP attracts dendritic |
| | possibly | hours | NPS | | cells. Emitted from dying |
| | in blood | before | | | tumor cells. ATP is an early |
| | | NPS | | | biomarker with greater |
| | | | | | increase at 4 hours. |
| HMGB1 | tumor, | 48-0 | 4-24 hours after | ≥35% increase | HMGB1 activate dendritic |
| | possibly | hours | NPS | | cells/Mφ. Emitted from |
| | in blood | before | | | dying tumor cells. HMGB1 is |
| | | NPS | | | a late biomarker with greater |
| | | | | | increase at 24 hours. |
| CD40 | Tumor | 48-0 | 24-48 hours after | ≥35% increase | Confirm dendritic cell |
| | or | hours | NPS | | activation |
| | sentinel | before | | | |
| | lymph | NPS | | | |
| | nodes | | | | |
| CD80 | Tumor | 48-0 | 24-48 hours after | ≥35% increase | Confirm dendritic cell |
| | or | hours | NPS | | activation |
| | sentinel | before | | | |
| | lymph | NPS | | | |
| | nodes | | | | |
| CD86 | Tumor | 48-0 | 24-48 hours after | ≥35% increase | Confirm dendritic cell |
| | or | hours | NPS | | activation |
| | sentinel | before | | | |
| | lymph | NPS | | | |
| | nodes | | | | |
| MHC-II | Tumor | 48-0 | 24-48 hours after | ≥35% increase | Confirm dendritic cell |
| | or | hours | NPS | | activation |
| | sentinel | before | | | |
| | lymph | NPS | | | |
| | nodes | | | | |

If the biomarkers of interest have not exhibited the desired meaningful increases or decreases in concentration, then at block 514, a secondary treatment may be performed which would include re-application of sub-microsecond pulses of electric fields to the abnormal growth. For example, if MDSCs concentration has decreased less than 30%, more pulsing may need to be performed.

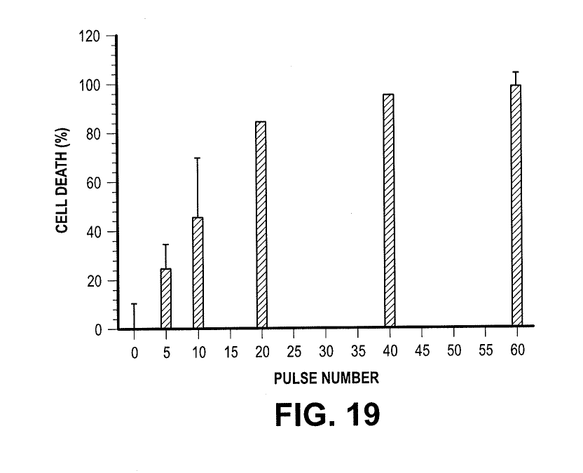

One result of the electrical pulse treatments administered in the studies is that a single treatment led to complete primary tumor regression in orthotopic breast cancer mice. This is in line with results of in vitro studies of the cytotoxicity of electrical pulses to 4T1 or 4T1-luc breast cancer cells, such as FIG. 19, which shows pulse number-dependent cytotoxicity of NPS for 4T1-luc cells (e.g., LD50 or LD99 for NPS with 60 ns, 1 Hz, 50 kV/cm is about 12 pulses and 45 pulses, respectively).

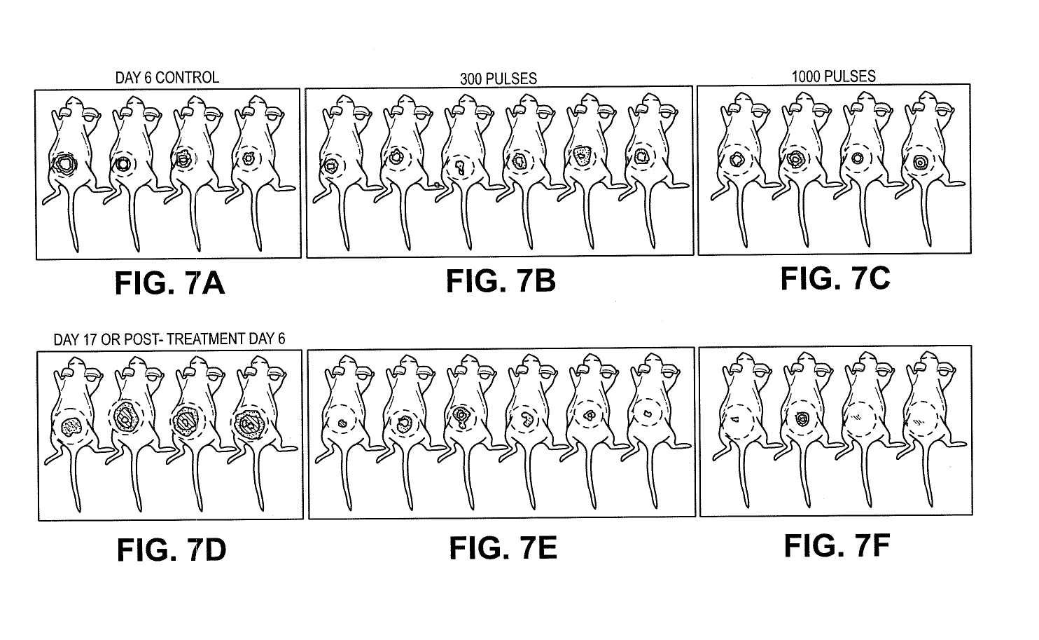

In the studies, the 4T1-luc tumors in the mice were treated with 100 ns, 1 Hz, 50 kV/cm and either 300 pulses or 1000 sub-microsecond electrical pulses for breast cancer elimination. FIGS. 6A-6D and FIGS. 7A-7F illustrate the regression of breast cancer in mice following a single treatment of administering sub-microsecond electrical pulses. More specifically, mice inoculated with 4T1-luc orthotopic breast tumors between the size of 5 to 7 mm were treated at day 11 with one of three methods: an administration of 0 (e.g., the control), 300, or 1000 sub-microsecond electrical pulses with 100 ns duration and 50 kV/cm at 1 Hz. The mice were then monitored for tumor growth or regression for the duration of the study (e.g., for 50 days from the mice being inoculated with the 4T1-luc tumors or until the tumor volume reached 1500 mm³). FIGS. 6A-6D display the tumor size over time for the three treatment groups based on various metrics (e.g., different techniques for measuring tumor size). In each of the graphs, day 11, the point in time at which treatment is administered, is indicated in the X-axis via the ‘Tx’ arrow.

In FIG. 6A, tumor volume is used as the indicator of tumor size and the 4T1-luc tumor volume over time is shown for the three treatment approaches. In order to determine tumor volume, a digital caliper is used twice a week to measure, for each tumor, the longest diameter of the tumor and the shortest diameter of the tumor perpendicular to the longest diameter. The tumor volume is then calculated using the formula V=πab²/6, where ‘a’ is the longest diameter of the tumor and ‘b’ is the shortest diameter of the tumor that is perpendicular to ‘a’.

In FIG. 6A, it can be seen that the tumor size is uniform across each treatment group on day 11 (when the treatments are administered) at approximately 100 mm³. Since no electrical pulses were administered to the tumor in the control group, the tumor size in the control group continues to grow steadily in size following day 11—eventually reaching a size of 1200 mm³ by day 35. In contrast, the administration of 300 pulses results in an initial reduction in tumor size following treatment, but it can be seen that the tumor is not completely eradicated. By day 25, the tumor exposed to 300 pulses continues to grow again and steadily reaches a size of 600 mm³ by day 50. Accordingly, the administration of 300 pulses is likely only enough to temporarily reduce the size of the tumor and delay its growth. However, for the group of mice that were treated with 1000 pulses, the tumor size decreases to 0 mm³ by day 25 and remains there for the duration of the study. This suggests that the application of 1000 pulses was sufficient to result in local tumor remission.

In FIG. 6B, luminescent biomarkers are used to stain the tumors for detection through in vivo imaging and the luminescence associated with each tumor, expressed as photons/sec/ROI (region of interest) minus background luminescence for a similarly-sized region, is used as the indicator of tumor size. From FIG. 6B, it can be seen that the tumors in all three treatment groups have a luminescence of about 5E+08 p/s on day 11 when the treatments are administered. However, from then onwards, the tumor luminescence for each treatment group begins to diverge. In the case of the control group, no electrical pulses are administered to the tumor so the tumor continues to grow in size, which is reflected as steadily increasing in luminescence up to day 35. Tumors exposed to 300 pulses exhibited a short-lived decline in luminescence following treatment, but by day 15, the luminescence continues to increase steadily until day 50 when the study concludes. This matches the trend associated with measured tumor volume for tumors exposed to 300 pulses, as shown in FIG. 6A, thereby providing additional evidence that 300 pulses results in temporary tumor regression but is insufficient for totally eradicating the tumor. Finally, for tumors exposed to 1000 pulses, the luminescence for those tumors experiences a steep drop-off into day 25, at which point the luminescence bottoms out at approximately 1E+05 p/s. This also suggests that the application of 1000 pulses was sufficient to result in local tumor remission.

FIG. 6C further breaks down the results specific to the 300 pulse (e.g., low dose) treatment group. In particular, the figure displays in vivo imaging data for the tumor luminescence associated with each mouse in the 300 pulse treatment group. From the figure, it can be seen that the luminescence curve associated with the 300 pulse treatment group displayed in FIG. 6B did not tell the whole story. The tumor luminescence for two of the mice treated with 300 pulses dropped steadily following treatment and reached 1E+05 p/s by day 25, while the tumor luminescence for the remaining mice treated with 300 pulses only dropped temporarily before continuing to increase into day 50. This suggests that the 300 pulse treatment is inconsistent at totally eradicating the tumor in every subject, but it will consistently cause a temporary regression in the tumor.

FIG. 6D further breaks down the results specific to the 1000 pulse (e.g., high dose) treatment group. In particular, the figure displays in vivo imaging data for the tumor luminescence associated with each mouse in the 1000 pulse treatment group. It can be seen that the luminescence curve for each mouse closely mirrors the overall luminescence curve associated with the 1000 pulse treatment group displayed in FIG. 6B. In particular, the tumor luminescence for all of the mice treated with 1000 pulses steadily drops following treatment to reach 1E+05 p/s by day 25, a level at which the luminescence remains at for the remainder of the study. Accordingly, this suggests that the 1000 pulse treatment is consistent eradicating the 4T1-luc tumor.

FIGS. 7A-7F are pictures of in vivo imaging on days 6 and 17 after tumor inoculation for each of the three treatment groups (control, 300 pulses, and 1000 pulses). More specifically, FIGS. 7A-7F show the luminescent tumors for each mouse in all three treatment groups on days 6 and 17 after tumor inoculation. There are is a total of 4 mice in the control group shown in FIGS. 7A & 7D, a total of 6 mice in the 300 pulse treatment group shown in FIGS. 7B & 7E, and a total of 4 mice in the 1000 pulse treatment group shown in FIGS. 7C & 7F.

For the control group shown in FIGS. 7A & 7D, the increased luminescence from day 6 (FIG. 7A) to day 17 (FIG. 7D) shown in each mouse reflects the increase in size for the tumors in the four mice of the control group over that time. Without any kind of treatment, the tumors continue to grow following inoculation.

For the 300 pulse treatment group shown in FIGS. 7B & 7E, the luminescence for two of the mice shown in FIG. 7B (the mice on each end) greatly decreased from day 6 (FIG. 7B) to day 17 (FIG. 7E), which suggests that the tumors in those mice steadily regressed throughout that time period. This matches the observations from FIG. 6C, which shows the luminescence associated with two of the mice in the 300 pulse treatment group dropping steadily following treatment. For the other four mice in the 300 pulse treatment group shown in FIGS. 7B & 7E, the luminescence appears to only have decreased slightly. This is also reflected by FIG. 6C, which shows a temporary dip in luminescence for those four mice between day 6 to day 17 before the luminescence starts increasing again. If there were additional imaging taken from the period of day 17 to the conclusion of the study (e.g., day 50), it is likely that the tumors in these four mice would be shown to continue to grow following day 17.

For the 1000 pulse treatment group shown in FIGS. 7C & 7F, the luminescence for all of the mice greatly decreased from day 6 (FIG. 7C) to day 17 (FIG. 7F), which suggests that the tumors in those mice steadily regressed throughout that time period. This matches the observations from FIG. 6D, which shows the luminescence associated with all of the mice in the 1000 pulse treatment group dropping steadily following treatment until luminescence bottoms out. Even after day 17, the luminescence continues to stay low across each of those mice which suggests the tumors were eradicated by the application of 1000 pulses.

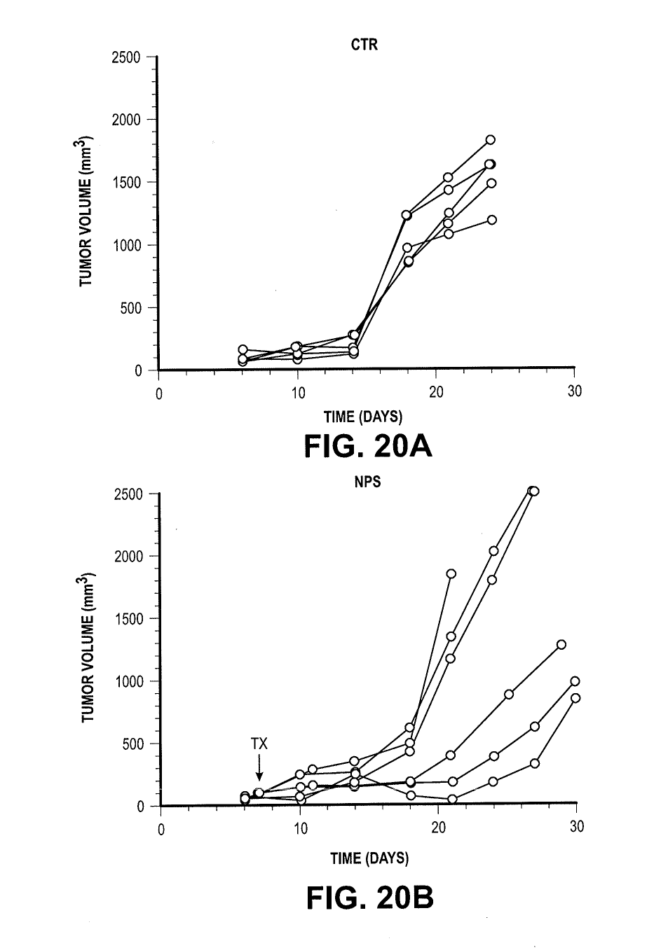

To summarize, from FIGS. 6A-6D and 7A-7F, when using a five needle electrode and a high number of NPS pulses (e.g., 1000 pulses), a single treatment eradicated 100% of cancer in mice (i.e., 4/4 mice) without recurrence for 7 weeks when the study was terminated (graph 608). In contrast, lower numbers of NPS pulses (e.g., 300 pulses) only eliminated 33% of 4T1-luc tumors in mice (2/6 mice, graph 606). In either case, complete tumor regression could occur post-procedure as early as 3 days or as long as 10 to 13 days. Because of scar formation, the caliper measurements were not an adequate indication of real tumor regression within 10 days after treatment, especially for 1000 pulse treatment group (FIG. 6A and FIG. 7C). These results may be somewhat dependent on electrical pulses and electrodes having certain parameters. For instance, in additional studies, a two-needle electrode or a two-plate suction electrode with the same 5 mm gap was used. However, no complete tumor regressions (0/7 mice, as seen in FIG. 20B) were achieved by using the two-needle electrode at 600 pulses or 2×600 pulses by applying electrodes in successive perpendicularly applications in the same treatment session.

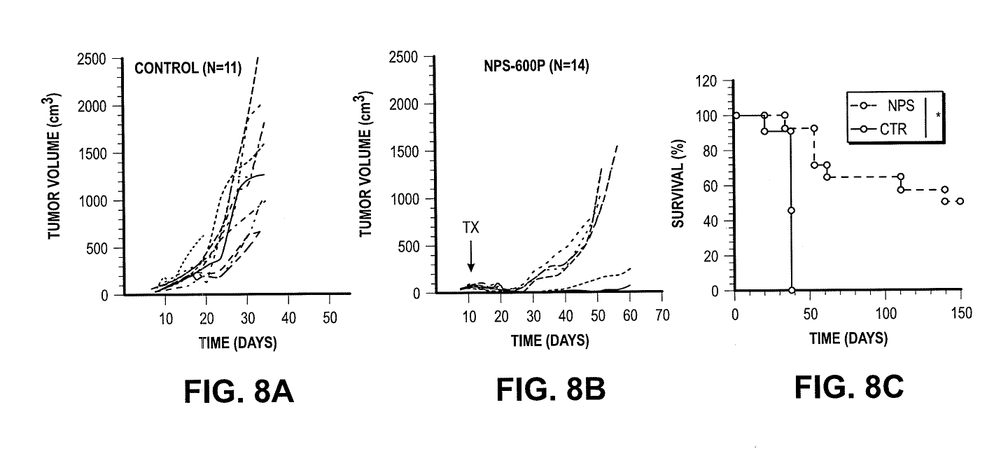

Another result of the electrical pulse treatments administered in the studies is that even incomplete local tumor ablation after NPS treatment prevented/attenuated distant metastases, which is a surprising find considering electrical pulse stimulation is perceived as a physical modality of treatment. This result is reported in FIGS. 8A-10B.

FIGS. 8A-8C illustrate how breast cancer metastases in mice are prevented following the treatment of a local tumor with sub-microsecond duration electrical pulses. FIGS. 8A-8C are different from FIGS. 6A-6D and 7A-7F, which display the changes in local tumors that were directly treated with electrical pulses. In contrast, FIGS. 8A-8C focus primarily on the indirect changes in metastases resulting from administering sub-microsecond duration electrical pulses to a tumor at an entirely separate location. Although the results in FIG. 8 are associated with metastases in mice, similar results should be observed in humans with late stage breast cancer because of its similarities to the metastatic 4T1-luc mouse mammary carcinoma used to inoculated the mice.

FIGS. 8A-8B display, respectively, the tumor volume of the metastases for the mice in two separate treatment groups: a control group and a group treated with 600 pulses. As in FIGS. 6A-6D, the tumor volume is used as an indicator of tumor size. To determine tumor volume, a digital caliper is used twice a week to measure, for each tumor, the longest diameter of the tumor and the shortest diameter of the tumor perpendicular to the longest diameter. The tumor volume is then calculated using the formula V=πab²/6, where ‘a’ is the longest diameter of the tumor and ‘b’ is the shortest diameter of the tumor that is perpendicular to ‘a’.

There is a total of 11 mice in the control group and a total of 14 mice in the group treated with 600 pulses of 100 ns in duration and 50 kV/cm at 1 Hz via a two-plate suction electrode with a 5 mm gap. Only an intermediate number of pulses is applied here to avoid complete tumor regression, since the 4T1-luc tumor model is metastatic and it is of interest of the study to compare levels of distant metastasis in control and incompletely treated tumors. All of the mice were inoculated with orthotopic breast tumor and treatment (for the mice in the treatment group) was administered on day 12 following inoculation, which is indicated in the X-axis of FIG. 8B with the ‘Tx’ arrow. The treatment takes 5 minutes for delivery of 600 pulse at 2 Hz and was stopped part way through re-apply gel to ensure that it is not removed by the suction during treatment. Like previous studies treating ectopic tumors, scabs form after treatment but are resolved within two weeks.

FIG. 8A displays the tumor volume of metastases for mice in the control group. From the figure, it can be seen that, in each of the mice in the control group, the tumor volume grows in size geometrically following inoculation. Some of the tumors reached a volume as high as 2500 cm³ by day 30 following inoculation.

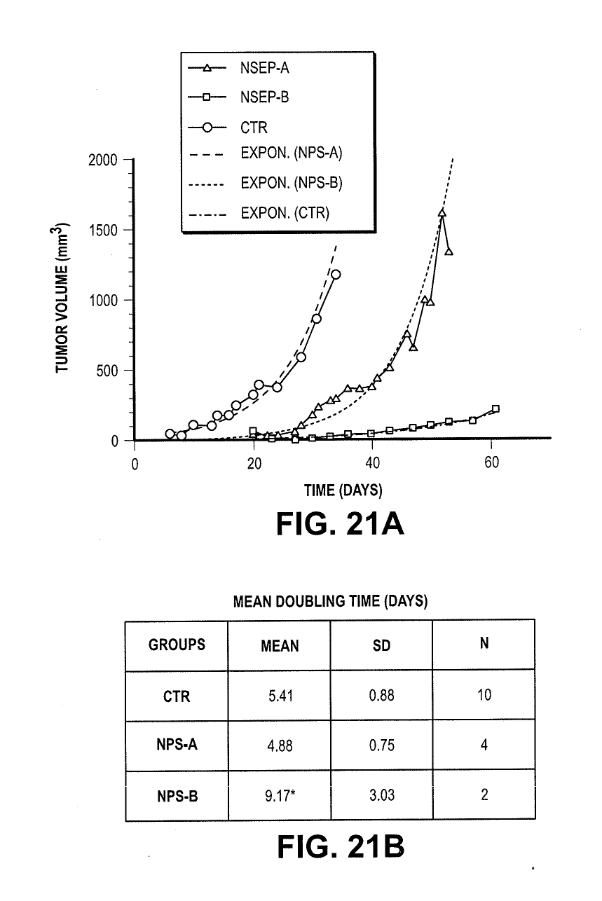

In contrast, FIG. 8B displays the tumor volume of metastases for mice in the treatment group. In seven of the mice in the treatment group, the tumor volume of the metastases drops to zero following treatment. However, this is not observed across all of the mice. For the remaining mice in the treatment group, the tumor volume experiences an initial decline for approximately 10-12 days before the tumor starts growing again. For two of those mice, the tumors were unmeasurable or greatly shrank in the second week post-treatment and re-grew, but remarkably slowly. The tumor doubling-times of those two mice were 50% longer than other NPS treated or control animals. This distinctive growth profile is further shown in FIGS. 21A-21B.

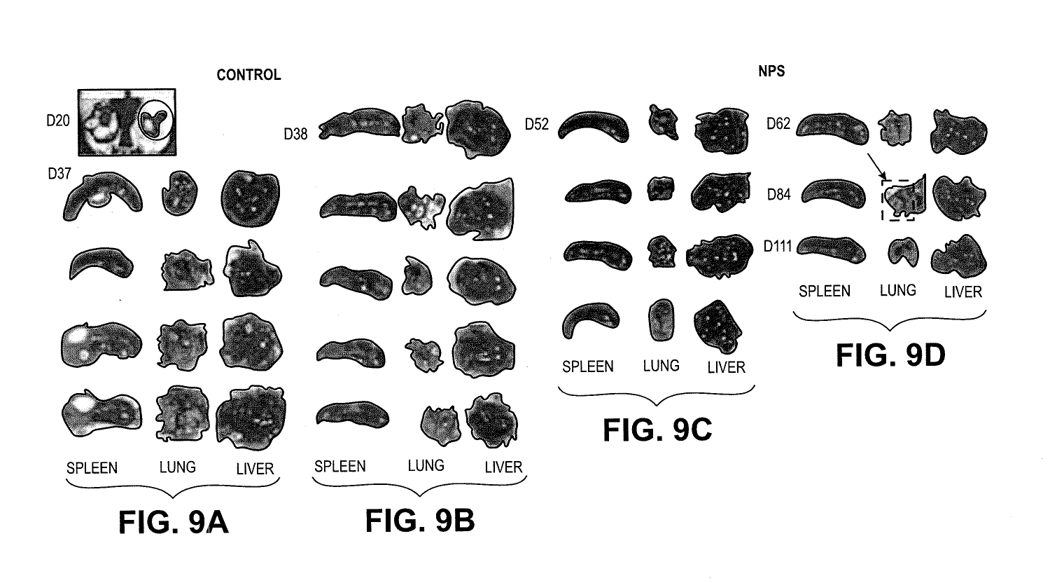

FIG. 8C displays Kaplan-Meier survival curves for both groups of mice, either treated with our without electrical pulses. It can be seen that the control group rapidly drops off to 0% survival (e.g., all the mice are dead) by day 40, with most of the mortalities occurring between day 30 and day 40. In contrast, for the group of mice treated with 600 pulses, the survival curve drops off at a much slower rate, and 90% of the mice are still alive by day 40. The survival rate eventually reaches 50% by day 150. In fact, 50% (7/14 mice) of the tumors completely regressed by a single treatment with 600 pulses (FIGS. 8B, 8C). The 50% tumor free rate, as measured via calipers, is confirmed by the in vivo imaging shown in FIGS. 9A-9D. Thus, graph 806 clearly shows how treatment with 600 sub-microsecond electrical pulses greatly prolongs the survival of mice inoculated with the 4T1-luc tumor. Median survival was significantly increased 25 days in the incompletely treated group compared to control animals (with a significance of p<0.001).

FIGS. 9A-9D are in vivo (at day 20) or in vitro (at days 37 and 38 for the control group, and at days 52, 62, 84, or 111 for the group of mice treated with electrical pulses) imaging for luciferase expression indicating organ metastases. The control and treated tumors of similar sizes were analyzed (4 weeks for controls and 7 weeks or longer for NPS-treated tumors). The organs shown in the images include the spleen, lung, and liver of mice that had large tumors at the endpoint of the study based on IACUC protocol.

More specifically, FIGS. 9A-9B illustrate the spleen, lung, and liver of the mice in the control group at days 37 and 38, with any metastases stained and visible. In FIG. 9A, it can be seen that by day 37, the tumor has consistently metastasized to the spleen, lung, and liver. FIG. 9B shows that by day 38, the tumor has also consistently metastasized to the lung. Distant metastases were found in liver, lung and/or spleen in 82% (9/11) of control mice.

FIGS. 9C-9D illustrate the spleen, lung, and liver of the mice in the treatment group at days 52 62, 84, or 111, with any metastases stained and visible. The mice in the treatment group were treated with a two plate suction electrode that administered 600 electrical pulses of 100 ns, 50 kV/cm, 1 Hz. Strikingly, six of seven mice (86%) with incomplete tumor regression (p=0.013 vs control) exhibited no detectable metastases in liver, lung or spleen by luminescent ex vivo imaging of isolated organs. FIG. 9C shows that by day 52, none of the cancer has metastasized to the spleen, lung, or liver. FIG. 9D shows that by days 62, 84, and 111, the cancer still has not metastasized to the spleen, lung, or liver for the most part. The singular exception is indicated by an arrow that corresponds to a metastasis in the lung on day 84.

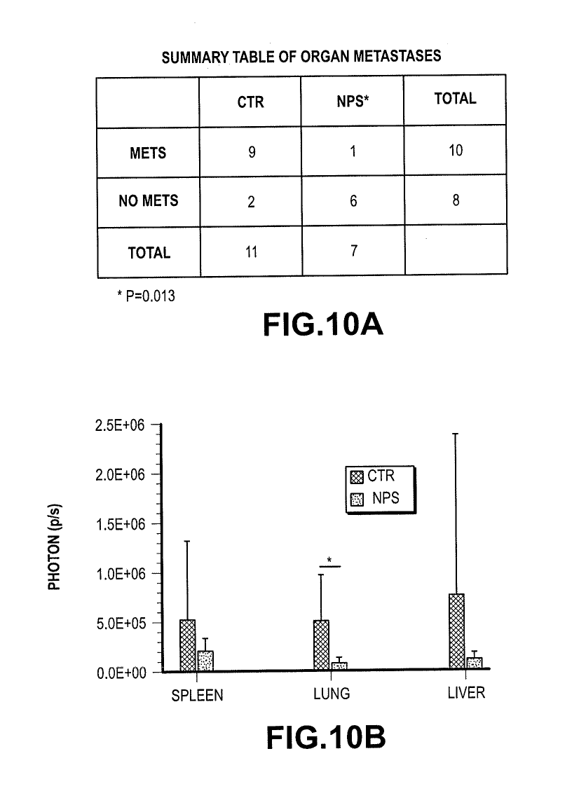

FIGS. 10A-10B illustrate quantified data for the organ metastases (e.g., associated with FIGS. 8A-8C and 9A-9D). In particular, FIG. 10A provides a summary table of organ metastases from the control group and the treatment group. In the control group of 11 mice, there was a total of 9 mice with metastases and 2 mice with no metastases. In the treatment group, there was a total of 1 mouse with metastases and 6 mice with no metastases. For the treatment group, these results have a significance of p=0.013 (Chi-square test).

FIG. 10B quantifies the photons (p/s) received (e.g., the luminescence associated with the metastases) from the organ metastases through in vitro imaging. For the control group, where no electrical pulses were administered to the mice, the mice had significantly higher amounts of photons received from the spleen, lung, and liver than the treatment group, which suggests that there are more cancer cells present in the spleens, lungs, and livers of the control group. These results have a significance of p=0.004 (t-test).

The observations from FIGS. 8A-10B are surprising, since comparisons of metastasis between the control group (shown in FIGS. 9A-9B) with the treated group of (shown in FIGS. 9C-9D) were made when tumor sizes were equivalent (and thus, the metastasis is expected to be equivalent). Thus, there must be some mechanism involved in the treated group that is preventing metastasis from occurring. This mechanism likely involves antitumor immunity induced by the electrical pulse treatment, which can be confirmed by the presence of long term antitumor immune memory and the local and systemic immune response after treatment.

Thus, steps were taken to observe the presence of long term antitumor immune memory and the local and systemic immune response after treatment.

FIGS. 11A-15 report the data associated with the findings and reveal how successful NPS treatment can surprisingly result in establishment of a potent anti-tumor memory response in tumor-free mice that can reject secondary tumor challenge.

FIGS. 11A-11B illustrate how tumor-free mice that received electrical pulse treatment are protected from secondary tumor challenge (e.g., where the mice are inoculated again). More specifically, the figures display in vivo imaging after secondary tumor challenge at day 10 for two different groups of mice, one of which includes naïve age control mice and the other of which includes tumor-free mice following electrical pulse treatment. Both groups of mice were challenged intra-mammarily with 0.5 million 4T1-luc cells in the opposite mammary gland at least seven weeks after the initial treatment of the tumor-free mice with electrical pulses. There are 11 tumor-free mice following electrical pulse treatment shown in FIG. 11A and 9 naïve age control mice shown in FIG. 11B. By comparing the luminescence observed in the mice between the figures, it can be seen that the size of the tumors from secondary tumor challenge are much bigger in the control group compared to the tumor-free group.

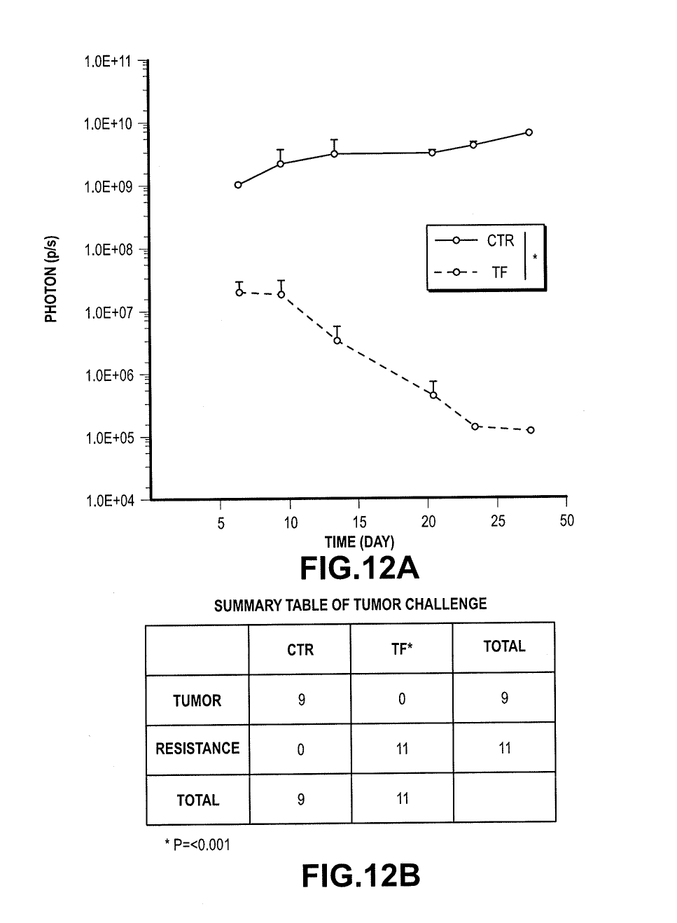

FIG. 12A-12B provide quantitative data associated with the in vivo imaging shown in FIGS. 11A-11B to further illustrate the point that pulse-treated, tumor-free mice are protected from secondary tumor challenge. More specifically, FIG. 12A illustrates the photons (p/s) received (e.g., the luminescence associated with the site of secondary tumor challenge) for the two groups of mice (control and tumor-free) following secondary tumor challenge on day 10. For the control group, the photons received steadily increases in the days following secondary tumor challenge, which reflects growth of the secondary tumor. In contrast, the photons received rapidly drops for the pulse-treated, tumor-free mice after day 10. By day 30, the photon level has dropped to 1.0E+05. This reflects the regression of the secondary tumor, likely due to the resistance provided by electrical pulse treatment (e.g., by stimulating the immune response and the other mechanisms previously discussed herein). These results have a significance of p=<0.001 (t-test for the AUCs of two groups).

FIG. 12B provides a summary table of the secondary tumor challenge. Of the 9 total age-matched naïve mice in the control group, all 9 developed the secondary tumor and none had resistance. This is reflected by FIG. 12B, which depicts the control mice growing large tumors and having to be euthanized in five to six weeks post-injection. Of the 11 total mice in the pulse-treated, tumor-free group, none developed the secondary tumor and all had resistance. As a matter of fact, not even transient solid tumors were formed at the challenge injection sites in majority of tumor-free mice. Only red, soft inflammatory spots were observed one week after challenge injections, with complete resolution within two to three weeks. These results have a significance of p=<0.001 (Chi-square test).

Like the diminution of metastasis (e.g., FIGS. 8A-10B), protection from challenge injections (FIGS. 11A-12B) suggested an immune memory response. This can be further confirmed by analyzing phenotypes of CD4+ and CD8+ T-cells for markers of effector and central memory cells in blood and spleen of control and treated mice.

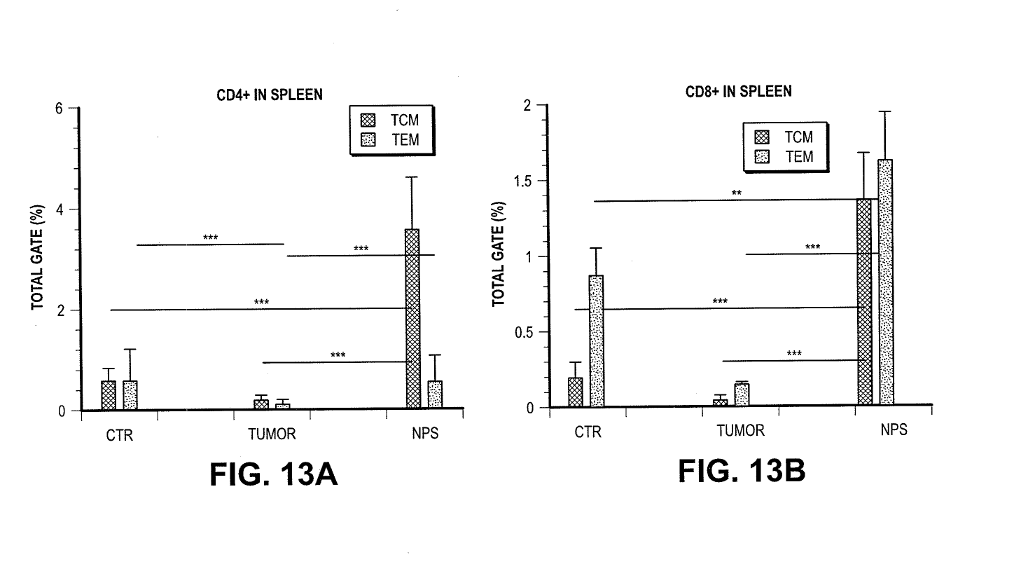

FIGS. 13A-13B illustrate the induction of long-term memory T cells and antitumor immunity after electrical-pulse treatment, which helps to explain the protection from secondary tumor challenge provided by electrical-pulse treatment as demonstrated in FIGS. 11A-11B and 12A-12B. FIGS. 13A-13B provide the levels of CD4+(FIG. 13A) and CD8+(FIG. 13B) memory T cells in the spleens of mice from three groups: a control group of cancer-free mice, a tumor group of mice having the tumor, and a NPS group of mice that are now tumor-free following electrical pulse treatment. The levels of CD4+ and CD8+ are reported as total gate (%), with ‘Tcm’ referring to the central memory T cells of that type and ‘Tem’ referring to the effector memory T cells of that type. It can be seen that compared to the control, the tumor group exhibits lower levels of both CD4+ and CD8+ memory T cells due to the tumors suppressing the immune systems of the mice. The NPS group has drastically elevated levels of CD4+ memory T cells as compared to the tumor group. The NPS group also has greatly higher CD4+ central memory T cells than the control, although the levels of CD4+ effector memory T cells are similar. Similarly, the NPS group has drastically elevated levels of both types of CD8+ memory T cells as compared to both the tumor group and the control group.

In other words, compared to control mice, both effector and central memory CD8+ and CD4+ T cells in the spleen and blood were significantly depressed in tumor-bearing mice. However, both of CD4+ and CD8+T memory cells in the blood or spleen of treated, tumor-free mice were recovered or surpassed levels of control mice, especially central memory CD4+ and CD8+ T cells. Memory T cells in spleens were greatly increased in the tumor-free mice compared to naïve control mice with CD4+(3.61% vs. 0.62%) and CD8+ T-cells (1.38% vs. 0.2%) and compared to tumor-bearing mice with CD4+(3.61% vs 0.22%) and CD8+(1.38% vs 0.06%) T-cells (all comparisons, p<0.001). Thus, it can be concluded that successful electrical pulse treatment can induce the production of long-term memory T cells.

FIGS. 13C-13H provide a representative histogram of intracellular cytokine staining for each group of mice after 6 hours incubation with plate bound anti-CD3. For instance, FIGS. 13C & 13F show IFN-γ producing CD4+ and CD8+ T cells from the control group, FIGS. 13D & 13G show IFN-γ producing CD4+ and CD8+ T cells from the tumor group, and FIGS. 13E &13H show IFN-γ producing CD4+ and CD8+ T cells from the tumor-free group. After re-stimulation with anti-CD3 antibody for 6 hours, IFN-γ producing CD4+(0.242%) and CD8+ T cells (0.364‰) from spleens of tumor-free mice were significantly increased whereas IFN-γ producing T cells from tumor-bearing mice (0.029% for CD4+ and 0.031% for CD8+ T cells) were largely decreased in contrast to naïve control mice (0.135% for CD4+ and 0.116% for CD8+ T cells). The significant decrease of cytotoxic T cells in tumor-bearing mice can be explained with immune suppressive status from the tumor microenvironment.

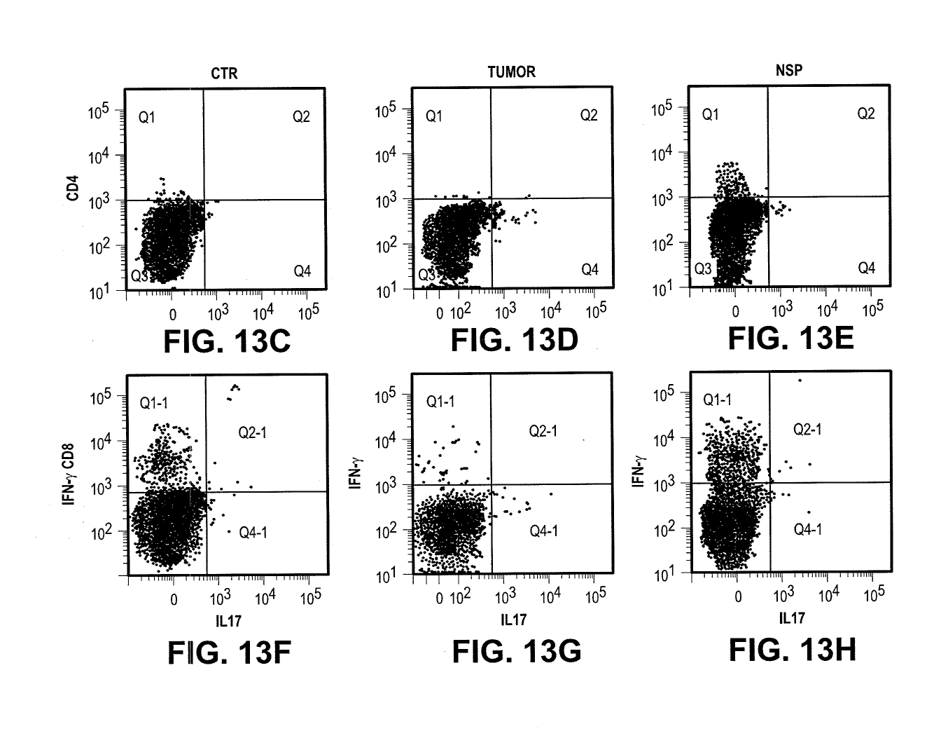

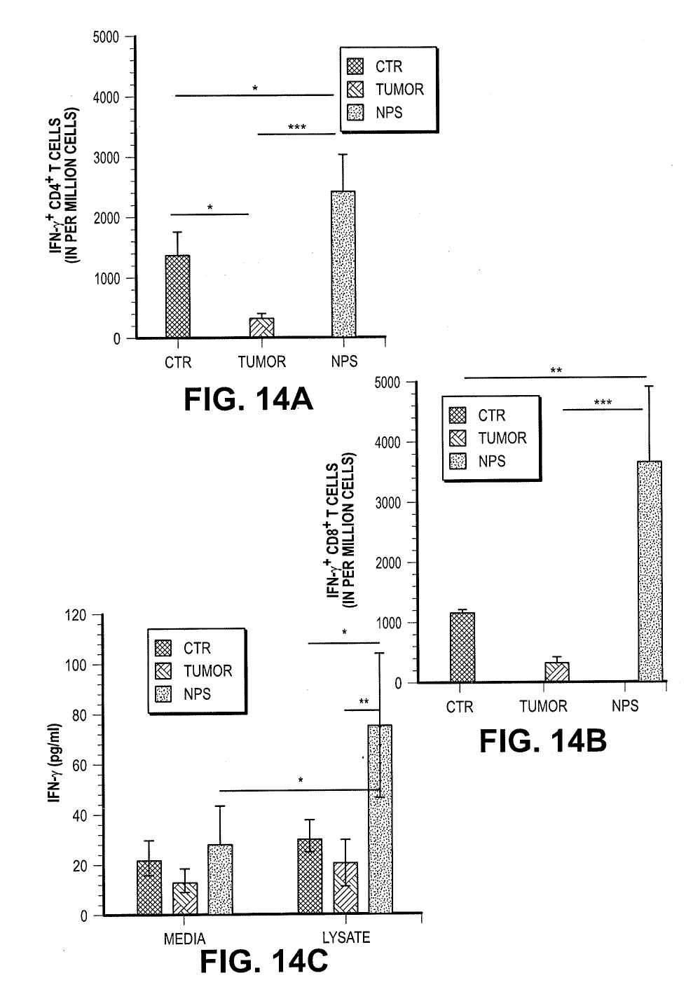

FIGS. 14A-14C illustrate the amounts of IFN-γ producing cells and IFN-γ product from splenocytes for the three groups of mice associated with FIGS. 13A-13H (e.g., a control group of cancer-free mice, a tumor group of mice having the tumor, and a NPS group of mice that are now tumor-free following electrical pulse treatment), and thus further help to explain the protection from secondary tumor challenge provided by electrical pulse treatment as demonstrated in FIGS. 11A-12B. There are 3 naïve mice at the age of 15-16 weeks in the control group, 4 mice at the age of 15-16 weeks and the end point for euthanasia in the tumor group, and 5 mice in the NSP group that are tumor free for at least 4 months after electrical pulse treatment.

FIG. 14A-14B illustrates the amount of IFN-γ producing cells from splenocytes after 6 hours incubation with plate bound anti-CD3. The amount of IFN-γ producing cells is higher in the NPS group compared to the amount of the control group, which is in turn higher than the amount in the tumor group.

FIG. 14C illustrates the level of IFN-γ product of splenocytes after 24 hours incubation with tumor lysate for the three groups. The level of IFN-γ product is highest for the NPS group of tumor free mice. The significantly high amount of IFN-γ was released from splenocytes of tumor free mice (75.9 pg/ml vs 28.5 pg/ml in the media control, p=0.021) in response to tumor antigens for 24 hours but not in the naïve mice (31.5 pg/ml vs 23.0 pg/ml in the control media, p=0.19) or tumor mice (21.1 pg/ml vs 11.8 pg/ml in the control media, p=0.114). In FIGS. 14A-14C, the stars represent the level of significance attached to the measurement: *: p<0.05, **: p<0.01, and ***: p<0.001 (One-way ANOVA).

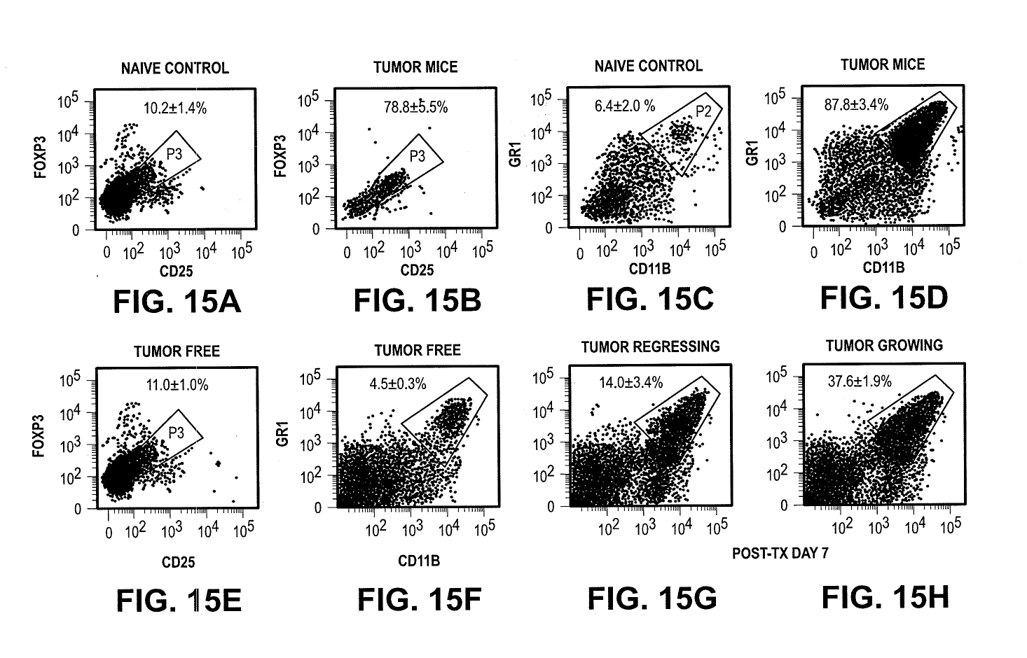

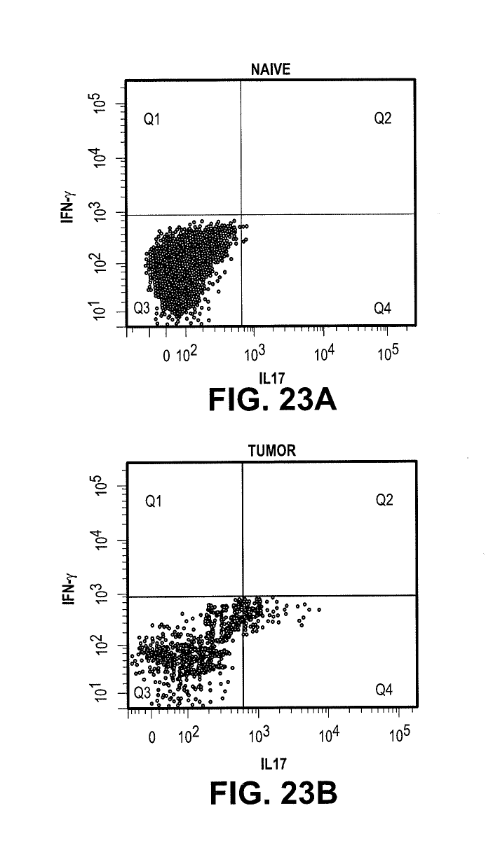

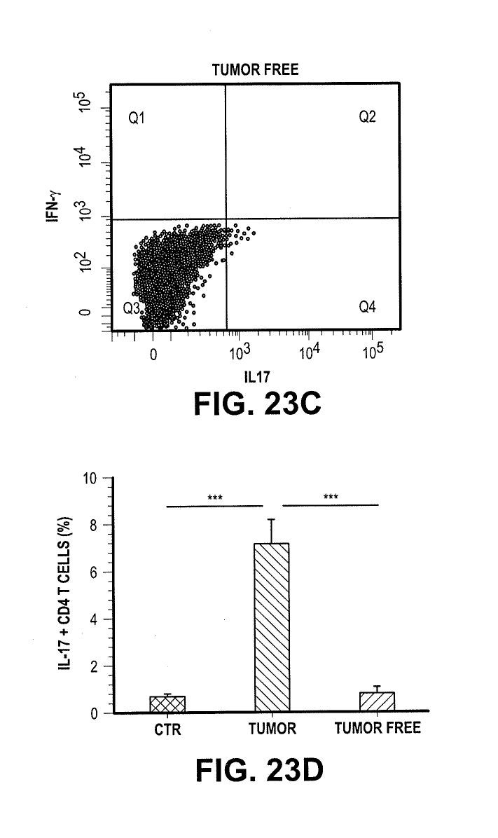

Additional confirmation of an established immune memory response can be attained from observing the local and systemic immune response after treatment. For instance, tumors and their surrounding microenvironment (e.g, tumor microenvironment) interact closely, with the tumor releasing extracellular signals, promoting tumor angiogenesis and inducing peripheral immune tolerance. The immune cells in the blood and tumor microenvironment can be suppressed to permit the growth and evolution of cancerous cells. The immune suppressive changes caused by 4T1-luc breast cancer can be observed in the hematopoietic and lymphatic systems of mice, resulting in splenomegaly (FIGS. 22A-22D), extremely high leukocytosis (FIGS. 22A-22D), myeloid derived suppressor cells (MDSCs) (FIGS. 15A-H), and IL-17 secreting cells (FIGS. 23A-23D). A reduction in this suppression of the immune cells can be observed following successful NPS treatment.

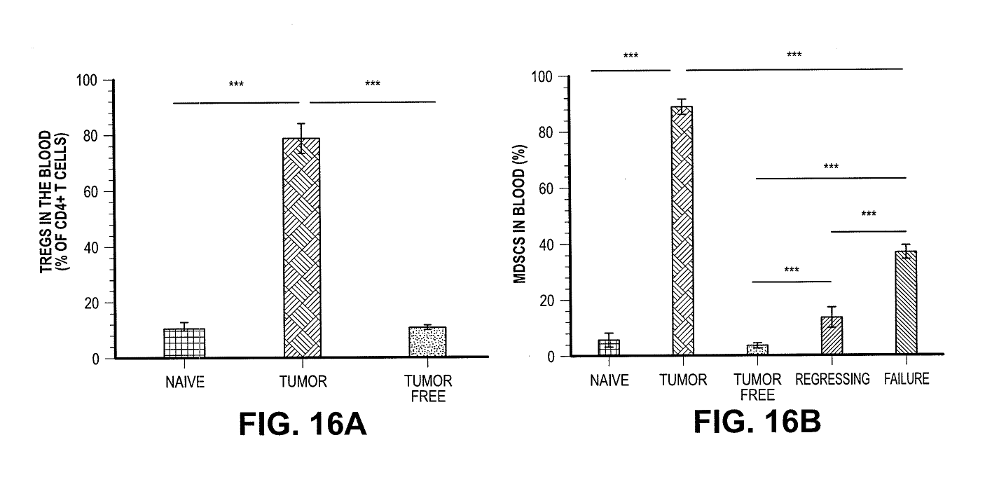

For instance, FIGS. 15A-16B illustrates the massive increase of immune suppressor cells in the blood of 4T1-luc breast cancer mice, and their reversal after electrical pulse treatment. This suggests that successful electrical pulse treatment reverses suppressive systemic immune environments (e.g., the blood in this case). FIGS. 15A-15H provide flow cytometry data associated with regulator T cells (Tregs) and myeloid derived suppressor cells (MDSCs) in the blood of naïve mice (n=5), tumor mice (n=6) or tumor free mice (n=4), nsEP treated mice while tumors were regressing (n=3) or growing (Failure, n=2) at post-nsEP treatment day 7. Specifically, FIGS. 15A & 15B & 15E provide flow cytometry data for FOXP3 and CD25 in the three groups (naïve control, tumor mice, and tumor-free), FIGS. 15C & 15D & 15F provide flow cytometry data for GR1 and CD11B in the three groups, and FIGS. 15G & 15H also provide flow cytometry data for GR1 and CD11B in the mice treated with nsEP while tumors were regression or growing. FIGS. 16A-16B provide a bar graph of summary data.

Tregs and MDSCs can be found in the blood and tumor microenvironment of a subject with cancer and play roles in suppressing the immune system and permitting the growth and evolution of cancerous cells. In the figures, 78.8% of CD4+ T cells were CD25+ Foxp3+ Treg cells in the blood of the tumor-bearing mice. In contrast, only 14.9% and 15.9% of CD4+ T cells were Treg cells in the tumor-free mice or control naive mice, respectively. Changes were examined for MDSCs as well. Compared to naive mice (6.4%) or tumor-free mice (4.5%), 13.7 fold or 19.5 fold increase of blood CD11b+Gr1+ MDSCs in tumor-bearing mice (87.8%) were measured by flow cytometry (p<0.001 between any two groups except naïve vs tumor free mice). Moreover, the level of MDSCs was correlated to success or failure of NPS treatment. At post-treatment day 7, mice with regressing tumor exhibited only 14.0% MDSCs in the blood. That was much lower than 37.6% MDSCs in the blood of mice with growing tumor or treatment failure. The decrease in Tregs and MDSCs in the blood suggests that successful electrical pulse treatment works to reverse suppressive systemic immune environments (e.g., by reducing immune suppression in the blood in this case).

The reduction in Tregs and MDSCs can also be found in the tumor microenvironments following treatment with electrical pulses. This is significant because MDSCs are known to accumulate in 4T1 mammary carcinoma bearing mice and present a barrier to the success of adoptive immunotherapy by suppressing T cell immunity.

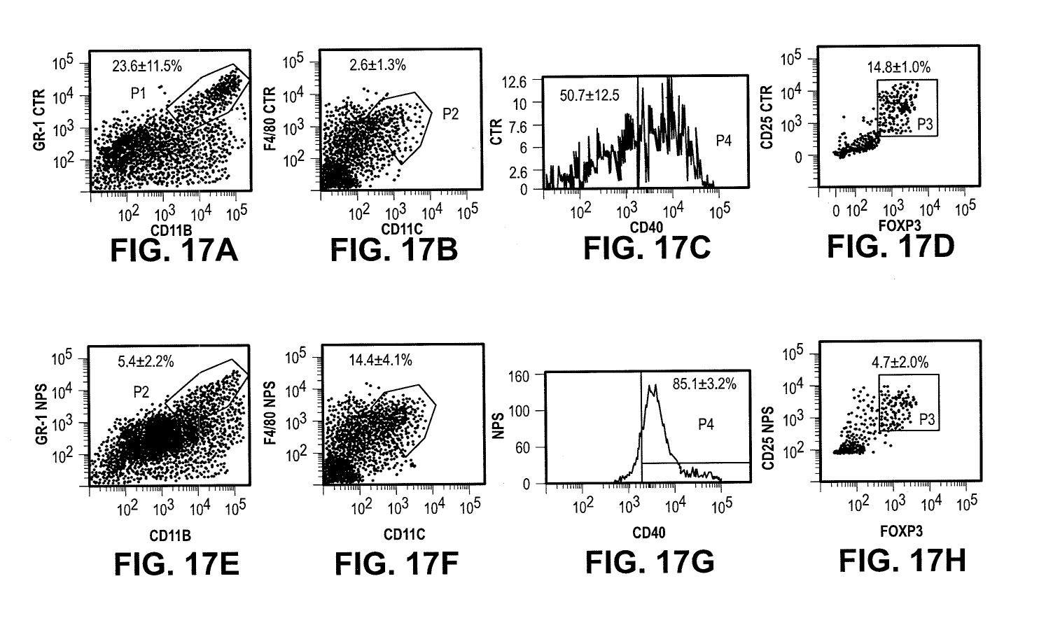

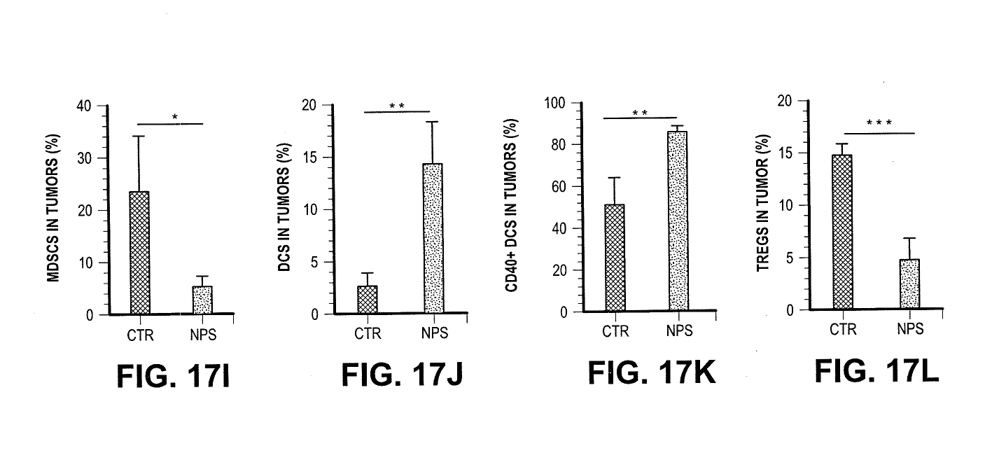

FIGS. 17A-17L show significant changes in tumor microenvironment after treatment with electrical pulses. For context, a control group of 3 mice with tumors were not administered treatment and a NPS treatment group of 4 mice with tumors were treated with electrical pulses. Tumors from both groups were collected (at post-treatment day 2 for FIGS. 17A & 17E & 17I, FIGS. 17B & 17F & 17J, and FIGS. 17C & 17G & 17K and at day 7 for FIGS. 17D & 17H & 17L) for single cell suspension preparation and cell labeling. The samples were fed into a flow cytometer in order to measure the amounts of various markers associated with the tumor microenvironment, such as markers for Myeloid-derived suppressor cells (MDSCs), dendritic cells (DCs), CD40+ dendritic cells, and regulatory T cells (Tregs). In particular, CD11B is a marker for MDSCs, CD11C is a marker for DCs, CD40 is a marker for CD40+ dendritic cells, and FOXP3 is a marker for Tregs.

FIGS. 17A & 17E show representative flow cytometer data of the amount of CD11B collected from the two groups. CD11B is a marker for MDSCs that allows the flow cytometer data to be plotted and quantified in terms of MDSCs. FIG. 17E is a bar graph of the amount of MDSCs in the collected tumors from the two groups. The MDSC percentage is about 23.6% for the control group and about 5.4% for the NPS treatment group. As a result of applying electrical pulses, the amount of MDSCs in the tumor site has drastically decreased. The significance of this result is p=0.017 (t-test).

FIGS. 17B & 17F show representative flow cytometer data of the amount of CD11C collected from the two groups. CD11C is a marker for DCs that allows the flow cytometer data to be plotted and quantified in terms of DCs. FIG. 17J is a bar graph of the amount of DCs in the collected tumors from the two groups. The DCs percentage is about 2.6% for the control group and about 14.4% for the NPS treatment group. As a result of applying electrical pulses, the amount of DCs in the tumor site has increased over five-fold. The significance of this result is p=0.005 (t-test).

FIGS. 17C & 17G show amounts of CD40 collected from the two groups. CD40 is a marker for CD40+ DCs that allows this data to be plotted and quantified in terms of CD40+ DCs. FIG. 17K is a bar graph of the amount of CD40+ DCs in the collected tumors from the two groups. The CD40+ DCs percentage is about 50.1% for the control group and about 83.1% for the NPS treatment group. As a result of applying electrical pulses, the level of CD40+ DCs in the tumor size has increased. The significance of this result is p=0.003 (t-test).

FIGS. 17D & 17H show representative flow cytometer data of the amount of FOXP3 collected from the two groups. FOXP3 is a marker for Tregs that allows the flow cytometer data to be plotted and quantified in terms of Tregs. FIG. 17L is a bar graph of the amount of Tregs in the collected tumors from the two groups. The Tregs percentage is about 14.8% for the control group and about 4.7% of CD4+ cells for the NPS treatment group. As a result of applying electrical pulses, the level of Tregs in the tumor site has drastically decreased. The significance of this result is p=<0.001 (t-test).

These graphs of FIG. 17A-17L demonstrate that NPS treatment can induce immune responses by also reversing the suppression of the tumor microenvironment, initiating anti-tumor cytotoxic T cells and promoting effector and central memory T-cells.

In order to determine the mechanisms of how the immune responses are instructed, studies were conducted to understand how NPS-induced tumor elimination activates dendritic cells (DCs). In particular, 4T1 tumor cells were treated and the release of damage-associated molecular patterns (DAMPS) was observed. DCs in vitro were also treated with NPS and the expression of co-stimulatory molecules was observed.

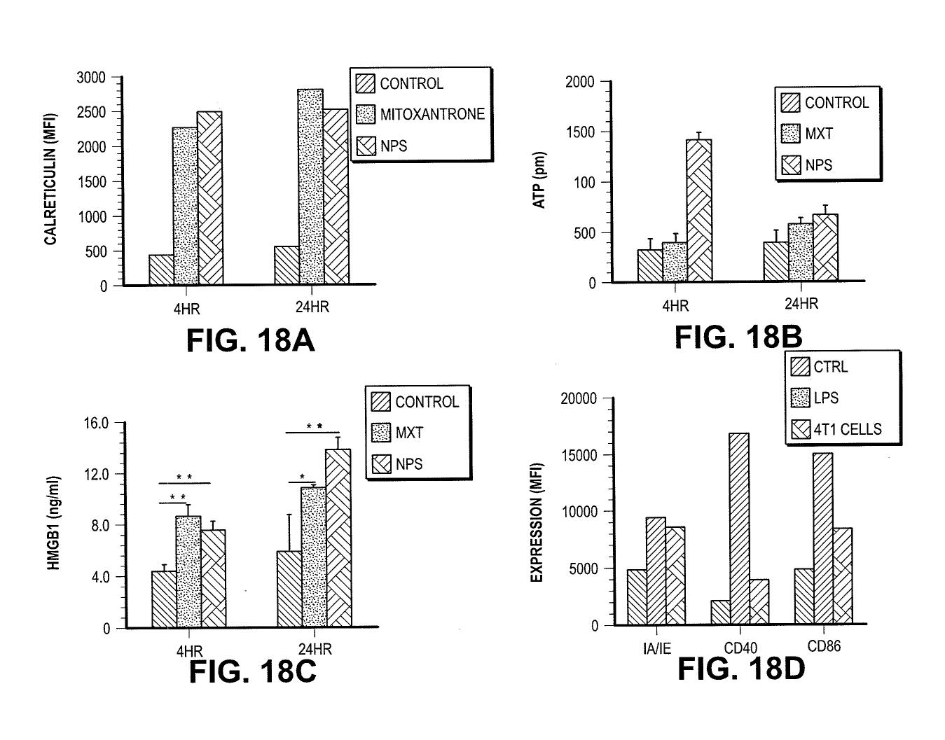

FIGS. 18A-18D illustrate the release of damage-associated molecular patterns (DAMPS) from pulse-treated 4T1 tumor cells and the activation of dendritic cells. The data from these graphs is associated with three groups of mice: a control group of mice without any treatment, a MXT group of mice treated by Mitoxantrone, and a nsEP group of mice that are treated with sub-microsecond duration electric pulses.

FIG. 18A shows the levels of calreticulin at 4 hours and 24 hours following treatment, as measured in mean fluorescence intensity (MFI). The MFI of calreticulin increases from 4 hours to 24 hours in all three groups, with the largest increase shown in the MXT group.

FIG. 18B shows the levels of ATP release in the supernatants at 4 hours and 24 hours following treatment, as measured in pM. ATP increases from 4 hours to 24 hours in the control and MXT groups. However, in the case of the mice treated with electrical pulses, the ATP drops from 1400 pM down to 650 pM in that time frame.

FIG. 18C shows the levels of HMGB1 release in the supernatants at 4 hours and 24 hours following treatment, as measured in ng/mL. HMGB1 increases from 4 hours to 24 hours in all three groups. However, HMGB1 increases the most for the mice treated with electrical pulses, with the HMGB1 increasing from about 8 to 14 ng/mL. The stars in the graph are associated with the significance of the results; *: p=<0.05, **: p=<0.01, ***: p=<0.001 (One-way ANOVA).

FIGS. 18A-18C are significant because calreticulin, ATP, and HMGB1 are known to be danger-associated molecular patterns (DAMPs). It is known that the release of several DAMPs from chemotherapeutic drug-induced cancer cell death can initiate anti-tumor immunity. Since cancer cells killed by NPS can release DAMPs such as calreticulin, ATP and HMGB1, it is possible that NPS can similarly initiate anti-tumor immunity. Supporting this is the showing that all three DAMPs from NPS treated 4T1 cells were greatly increased to a comparable level for calreticulin, and greater levels for HMGB1 and ATP, than cells treated with mitoxantrone, a known strong inducer of immunogenic cell death.

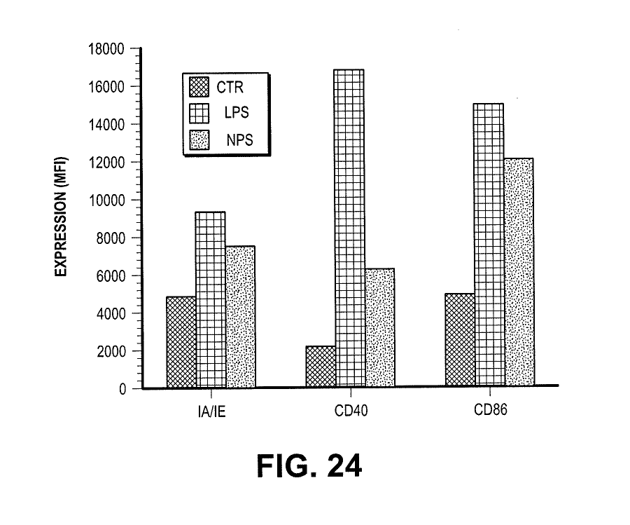

FIG. 18D shows the levels of co-stimulatory molecule (IA/IE, CD40, and CD86) expression for three types of dendritic cells (DCs), as measured in mean fluorescence intensity (MFI). The control type refers to dendritic cells (DCs) in media. The LPS type refers to DCs with 10 μg/ml LPS. The ‘4T1 cells’ type refers to DCs co-cultured with 4T1 cells and treated with electrical pulses. The DCs with 4T1 cells treated with electrical pulses show higher levels of all three co-stimulatory molecules as compared to the control, although those levels are lower than the levels of co-stimulatory molecules expressed by DCs with 10 μg/ml LPS. The figure shows that IA/IE and co-stimulatory molecules, CD86 and CD40 were up-regulated 2 days after DCs were co-cultured with NPS treated 4T1 cells under LD90 condition. These results support the hypothesis that tumor death by NPS activates antigen presenting cells. Moreover, DCs were activated after treatment with non-lethal dose of NPS (60 ns, 20 kV/cm, 1 Hz and 10 pulses) as well. As seen in FIG. 24, the expression of all three markers, IA/IE, CD86 and CD40 on DCs were increased.

FIG. 19A illustrates the dose response (e.g., cell death rate) of sub-microsecond duration electrical pulses for 4T1-luc cells. For the data shown in the graph, 4T1-luc cells (5×10⁶) were treated with a number of pulses of 60 ns, 50 kV/cm, and 1 Hz. The number of pulses used was variable, as indicated in the figure. At 5 pulses, about 25% of the cells died. At 10 pulses, about 45% of the cells died. At 20 pulses, about 85% of the cells died. At 40 pulses, about 90% of the cells died. At 60 pulses, about 95% of the cells died. The LD50 or LD99 for pulses with these parameters (60 ns, 1 Hz, 50 kV/cm) is about 12 pulses and 45 pulses, respectively. Thus, increasing the number of pulses improves the cell mortality rate, albeit with diminishing returns past a certain point.

FIGS. 20A-20B show the growth curve of a tumor with or without electrical pulse treatment having a different set of pulse and electrode parameters. In particular, FIG. 20A shows tumor volume (in mm³) over time for a control group of mice, while FIG. 20B shows tumor volume over time for a NPS group of mice treated with electrical pulses. To generate this data, the control group included 5 mice that were inoculated with the cancer cells and not provided any treatment. The NPS group includes 7 mice with the cancer cells that were treated on day 7 (following inoculation) with 600 electrical pulses of 100 ns, 50 kV/cm, and 1 Hz using a two-needle electrode. As seen in FIG. 20A, the tumor volume in the control group starts low but accelerates upwards past day 14, reaching between 1200 to 1700 mm³ by day 24. FIG. 20B shows that the tumor volume in the NPS group has delayed growth and begins to accelerate upward by day 18. No complete tumor regressions (0/7 mice) were achieved by using the two-needle electrode at 600 pulses or 2×600 pulses by applying electrodes in successive perpendicularly applications in the same treatment session. However, some of the mice that group have a distinct tumor growth pattern that results in much lower tumor volumes (e.g., volumes as low as 100-400 mm³) than the mice in the control group by day 24. Thus, the electrical pulse treatment can delay tumor growth and even reduce the rate of tumor growth in some cases of treatment. This can be seen more clearly in the next figures.

FIG. 21A and FIG. 21B show two distinctive growth patterns of tumors following electrical pulse treatment. This data is associated with three groups of mice, a control group of 10 mice without any treatment, a nsEP-A group of 4 mice that were treated with 100 ns, 50 kV/cm, 1 Hz and 600 pulses, and a nsEP-B group of 2 mice that were also treated with 100 ns, 50 kV/cm, 1 Hz and 600 pulses. The difference between the nsEP-A and nsEP-B groups is that the nsEP-A mice exhibited tumor growth with a similar pattern to that of the control mice, while the nsEP-B mice, following electrical pulse treatment, had tumors that grew much slower than the tumors of the control mice. For instance, FIG. 21A plots the tumor volume in (mm³) for the three groups of mice over the duration of a study. It can be see that the growth curve of the nsEP-A group is similar in growth rate to the growth curve of the control group, only the growth of the tumor is delayed by about 20 days due to the treatment (e.g., the nsEP-A growth curve is shifted over to the right by 20 days). However, for the nsEP-B group, the growth curve takes on a separate growth pattern in which the tumor grows much slower, reaching only about 200 mm³ in tumor volume by day 60.

The growth rates (e.g., mean doubling time) associated with these growth curves are quantified in FIG. 21B. For the control group, the mean doubling time is 5.41 days with a standard deviation of 0.88. The nsEP-A group has a similar growth rate, with a mean doubling time of 4.88 days and a standard deviation of 0.75. However, the nsEP-B group has a mean doubling time of 9.17 days with a standard deviation of 3.03. The significance of these results is *: p=<0.05 for nsEP-B to control or nsEP-A (One Way ANOVA).

FIGS. 22A-22D show splenomegaly and leukocytosis in 4T1-luc tumor burden mice as compared to a control group of naïve, tumor-less mice. The data used in this figure is associated with 8 tumor burden mice euthanized at the endpoint of the study and a naïve control group of 7 age matched mice without tumor. FIG. 22A, in particular, provides pictures of spleens from mice with tumors. FIG. 22B provides pictures of spleens from mice without tumors. It can be seen that the tumor spleens are enlarged in comparison (e.g., splenomegaly). FIG. 22C compares the weights (in mg) of the two different kinds of spleens shown in FIGS. 22A and 22B. The naïve, tumorless spleens have a weight of about 100 mg while the tumor spleens have a weight of about 800 mg. The significance of these results isp=<0.01. FIG. 22D compares the white blood cell counts (presented in millions) in the blood and spleen for the two different types of mice. For the tumor mice, the number of PBMCs and splenocytes is about 300 and 500 million, respectively. For the naïve control mice, the number of PBMCs is imperceptible on the graph while the amount of splenocytes is about 50 million. The significance of these results is p=<0.001 (t-test).

FIG. 23A-23D show the significant increase of IL-17+CD4 T cells (Th17) cells in the blood of tumors and a reversal to normal levels after electrical pulse treatment. FIGS. 23A-23C show flow cytometry data for IL17 (a marker for IL-17+CD4 T cells) associated with three groups of mice: a naïve control group of 3 mice without tumor, a tumor group of 3 tumor burden mice euthanized at the endpoint of the study, and a tumor free group of 6 mice that had complete tumor regression at least 49 days after being treated with 600 electrical pulses of 100 ns, 50 kV/cm, 1 Hz. FIG. 23D is a bar graph plotting the level of IL-17+CD4 T cells (as a percentage) between the three groups. The control group shows about 0.5% Th17 cells, a level which balloons to about 7% in the tumor group. It falls back down to around 0.5% in the tumor free group. Thus, the tumors cause a significant increase of Th17 cells which can return to normal levels following successful electrical pulse treatment and complete regression of the tumor. The significance of these results is p=<0.001 (One Way ANOVA). The reduction in the Th17 cells for the tumor free group further suggests that successful electrical pulse treatment reverses suppressive systemic immune environments (e.g., the tumor microenvironment).

FIG. 24 shows dendritic cell activation by electrical pulse treatment. Three types of dendritic cells (DCs) are represented in the data: a control group residing in media, a LPS group of DCs treated with LPS (10 μg/ml), and a NPS group of DCs treated with electrical pulses, 60 ns, 20 kV/cm, 1 Hz and 10 pulses. FIG. 24 is a bar graph plotting the expression of co-stimulatory molecules (as mean fluorescence intensity, MFI) from the DCs of the three groups, determined by flow cytometry after 48 hour incubation. The DCs of the control group expressed the least amount of co-stimulatory molecules (IA/IE, CD40, CD86). The DCs of the NPS group shows increased expression of all three co-stimulatory molecules. IA/IE increased by 2000 MFI, CD40 increased by 4000 MFI, and CD86 increased by 7000 MFI, all of which suggests that the DCs are activated due to the electrical pulse treatment.

From FIGS. 6A-24, it can be concluded that potent antitumor immunity is induced by local NPS tumor elimination. After NPS treatment, all eleven tumor-free mice (100%) rejected a second tumor challenge injection (FIGS. 11A-12B) that is consistent with a vaccine-like effect. Furthermore, for tumors that exhibited partial responses, NPS treatment resulted in a significant reduction in spontaneous distant organ metastases compared to untreated, control tumors of similar size. Thus, NPS treatment can provide a lasting benefit even if it is unsuccessful in bringing about complete tumor remission.

Furthermore, antitumor-specific IFN-γ production was increased in the NPS treated tumor-free mice. Consistent with challenge data and attenuation of metastasis, both CD4+ effector memory T-cells and CD8+ effector and central memory T-cells were significantly increased in tumor-free animals compared to control, naive mice or tumor-bearing mice (FIGS. 13A-14C). In addition, significant numbers of cytotoxic CD4+ and CD8+ T cells were detected in splenocytes from tumor-free mice in response to anti-CD3 stimulation.

Another impressive result of effective treatment with NPS is the reversal of immune suppressive tumor microenvironment in the 4T1-luc model. 4T1/4T1-luc breast cancer is a poorly immunogenic and highly spontaneous metastatic cancer model and very closely mimics human advanced breast cancer. The immunological characteristics of tumor-bearing mice were present here, including massive MDSCs and Tregs in the blood and tumor microenvironment, attenuation of cytotoxic T-cells and energy to immune stimulation or tumor antigens. All of these 4T1 characteristics provide evidence for the aggression of this mammary carcinoma model. Additional evidence for the highly metastatic potential in the 4T1 model is the powerful presence of MDSCs in the tumor microenvironment, which have been shown to be important for angiogenesis, a co-cancer hallmark with invasion and metastasis. NPS exhibits the impressive ability to treat such an aggressive disease by silencing the immune suppressive tumor microenvironment, a well-established therapeutic target that is recognized to play multiple roles in tumor progression, drug resistance, immune suppression, as well as angiogenesis and metastasis. Successful NPS treatment is able to bring about a reversal of the immune suppressive tumor microenvironment within two days after treatment, resulting in the diminution of angiogenesis and metastasis after treatment.

The results of the studies reported in FIGS. 6A-24 provide some clarity on the potential mechanisms through which NPS induces a potent antitumor immune response in a known poorly immunogenic cancer model merely from elimination of a local tumor. Some possible mechanisms are involved in the induction of anti-tumor immunity following NPS therapy: 1) tumor microenvironment destruction, as discussed above; and 2) ICD induction. ICD markers, including CRT, ATP or HMGB1, were released from NPS-treated 4T1 cells (FIG. 18A-18D). Bone marrow derived dendritic cells were activated and up-regulated MHC-II, co-stimulatory molecules CD86 and CD40 after co-culture with NPS-treated 4T1 cells (FIG. 18A-18D). The recruitment of DCs and CD40 upregulation was found in tumor after NPS tumor ablation as well. Another mechanism that may also participate is direct activation of antigen presenting cells by NPS. DCs can be activated (FIG. 24) with non-lethal doses of NPS, which likely occurs at peripheral area of tumor or in the normal tissue surrounding tumor where electric fields are below lethal levels.

Taken all together, first, NPS induces regulated cancer cell death (e.g., via apoptosis or necrosis), which releases immunogenic factors such as DAMPs (e.g., calreticulin, ATP, HMGB1) and tumor-associated antigens. At the same time, NPS also destroys the immunosuppressive environments (e.g., the immune-suppressive tumor microenvironment), reducing the levels of Tregs and MDSCs in the tumor microenvironment and blood. Second, NPS activates dendritic cells (DCs) both indirectly and directly. In the prior case, DCs are activated by the DAMPs (released by cancer cell death at the first step), loaded with the tumor-associated antigens, and recruited into the tumor tissue. Some DCs residing at peripheral tumor (where electric fields are below lethal levels) are directly activated by NPS, go into the tumor microenvironment, and induce the immune response. Third, some of the DCs migrate into lymphoid tissue and present antigens to T helper cells, with effector cytotoxic and memory T cells generated to establish a lasting immune response independent of the localized tumor site. This immune response in the periphery is sufficient to eliminate residual tumor cells, eliminate micro-metastases from establishing themselves, and prevent reoccurrence, even if the primary tumor has not been completely eradicated. In other words, the immune response not only prevents tumors at a nearby challenge site (e.g., to the primary tumor), but it also reduces tumors at distant sites.

In some embodiments, the low electric fields at the periphery of the tumor may also activate macrophages in addition to DCs. Thus, the electrical pulses may be of certain amplitudes sufficient to activate dendritic cells and/or macrophages at the periphery of the tumor. The immune-suppressive tumor microenvironment may normally prevent these macrophages from entering the tumor site or turn off the macrophages even if they enter the tumor site. By reducing the tumor microenvironment and activating the DCs in the periphery, the macrophages can more easily move into the tumor site and not be switched off. When they become activated, macrophages release ATP as part of activation process which may be reflected in the increased level of ATP in the blood or tumor microenvironment.

The antitumor immunity induced by NPS does not require any additional systemic/local immunotherapy, which is distinguishable from other local tumor ablation therapies that may only grant modest antitumor immunity if combined with systemic/local immunotherapy. For instance, hyperthermia by radio frequency ablation (RFA) of hepatocellular carcinoma or metastatic liver cancer was reported to induce antitumor immunity, but requires additional therapy, such as the addition of anti-PD-1 antibodies to achieve prolonged survival. Furthermore, RFA alone has failed to induce anti-tumor specific immune responses in a 4T1 mouse breast cancer model, whereas the combination with local IL-7 and IL-15 injection was necessary to elicit effective immune responses. As another example, irreversible electroporation (IRE), another minimally invasive approach for tumor ablation currently being tested in clinics, when used treat local advanced pancreatic cancer often results in distant metastasis and local recurrence. This suggests that IRE alone cannot induce antitumor immunity or is not strong enough to eradicate residual cancer or distant micrometastasis. As yet another example, radiation is another major local cancer therapy that has been used for half century. The abscopal effect, which was proposed as immune response following radiotherapy, has been found in clinics, but only in rare cases.

Thus, this ‘global’ anti-tumor immunity induced by NPS treatment at a single, localized tumor (without requiring any additional systemic/local immunotherapy) is a unique outcome when considering the typically-observed effectiveness of other physical, localized treatment modalities in inducing anti-tumor immunity. This immune response not only prevents tumors at a nearby challenge site (e.g., to the primary tumor), but it also reduces tumors at distant sites, which is indicative of a fairly strong immune response. Attaining such a strong immune response cannot be achieved by increasing the electric field intensity. Instead, successful NPS treatment likely results in anti-tumor immunity by reducing the tumor microenvironment (and its accompanying immune suppressor cells) and activating dendritic cells (e.g., to induce drastic increases in long-term memory T cells) for a lasting immune response.

Although NPS has been studied as a cancer therapy in several cancer models for localized tumor elimination with varying pulse and electrode parameters (for instance, pulse durations of 7 ns to 600 ns, electric field amplitudes of 10-68 kV/cm, pulse number 50 to 2700, and frequency 0.5 to 7 Hz, and different electrode designs such as needle electrodes with 2, 4, 5 needles, parallel plate electrodes, and suction electrodes), none of those studies have comprehensively evaluated the use of NPS for treating metastases or inducing antitumor immunity. However, the findings disclosed herein show that NPS can be used as a potent immunogenic cell death inducer to elicit anti-tumor immunity (in addition to its already-known use for local tumor eradication based on apoptosis/necrosis), which allows nanosecond electric pulse ablation to be used to treat metastatic diseases, e.g., advanced-stage cancers where the cancer has metastasized and multiple tumors are present.

In such applications, treatment with electrical pulses and electrodes having certain parameters discussed herein can be applied at a local tumor site in order to induce antitumor immunity and prevent distant metastases. The electrical pulse or electrode parameters may be different from those used to strictly treat a localized tumor. In some embodiments, the pulses may have a pulse duration, for example, of 100 to 600 ns. In some embodiments, the pulses may have a pulse duration of 7 to 300 ns. In some embodiments, the pulses may have a frequency of 1 to 7 Hz. In some embodiments, electric fields of 30-68 kV/cm may be used. In some embodiments, electric fields of 46-54 kV/cm may be used. In some embodiments, electric fields of 10-50 kV/cm may be used. In some embodiments, electric fields of 50 kV may be used. In some embodiments, 50-2700 electric pulses may be used. In some embodiments, a pulse number of up to 300, or between 300 and 600, or 1000 may be used. Different electrode designs may be used as well and associated with varying effectiveness based on the specific disease. For instance, a 6-pole dual electrode configuration has been known to reduce melanoma-GFP tumors in a nude mouse faster than a 6-pole single, a 2 pole or a 5 needle array. A 5 needle array electrode was better than a ring electrode in some cases, such as ectopic mouse HCC, orthotopic rat HCC, and treatment of orthotopic mouse mammary cancer as disclosed herein.

In some embodiments, the electrical pulses may be used in vitro rather than in vivo (e.g., to see if animals can be vaccinated by pulsing cells in vitro). Accordingly, in some embodiments, the pulses may have a duration of 20-100 ns. In some embodiments, the pulses may have a duration of 60 ns. In some embodiments, the electric field strength may be 20-60 kV/cm. In some embodiments, the electric field strength may be 40 kV/cm. In some embodiments, the number of pulses may be 1-20 pulses. In some embodiments, the number of pulses may be 1-10 pulses.

In some embodiments, the electrical pulses may have parameters (e.g., a sufficient electric field strength, number of pulses) that, when applied to the tumor, results in the expression of all three of calreticulin, ATP, and HMGB1. In some embodiments, the electrical pulses may have parameters that, when applied to the tumor, results in the expression of HSPC70, an immunogenic marker. In some embodiments, the electrical pulses may have parameters that, when applied to the tumor, activates dendritic cells. The activation of these dendritic cells may be confirmed by measuring the expression of HMGB1 and/or ATP.

In some embodiments, the electrical pulses may have parameters that, when applied to the tumor, causes a meaningful decrease of MDSCs or Tregs in the blood or tumor microenvironment. A meaningful decrease in this instance may refer to a 35% or greater decrease in MDSC or Tregs by two days or more following treatment. In some embodiments, the electrical pulses may have parameters that, when applied to the tumor, causes a meaningful increase in calreticulin, ATP, or HMGB1 in the blood or the tumor microenvironment. A meaningful increase in this instance may refer to a 35% or more increase in calreticulin at 6-10 hours following treatment, a 35% or greater increase in ATP at 12-24 hours following treatment, or a 35% or greater increase in HMGB1 at 24 hours or more following treatment. In some embodiments, the electrical pulses may have parameters that, when applied to the tumor, causes a meaningful increase in CD80, CD40, CD86, or MHC-II in the blood or tumor microenvironment. A meaningful increase in this instance may refer to a 20% or greater increase in CD80, CD40, CD86, or MHC-II at 24-48 hours following treatment. CD80, CD40, CD86, and MHC2 may serve as markers of dendritic cell activation.

In some embodiments, an agent may be introduced to the tumor cells just prior to electrical pulse treatment. For example, an epigenetic modulator or a phosphoinositide 3-kinase (PI3K) inhibitor can be injected into a microenvironment of the abnormal growth prior to electrical pulse treatment. PI3K would treat the tumor via one mechanism while the NPS would utilize a separate mechanism, and the combination of the two may yield synergistic results.

As stated above, according to another aspect of the present disclosure, devices and systems, including pulse generators and electrodes, configured to implement various methodologies disclosed herein, are provided. It will be appreciated that embodiments of the devices and systems of the present disclosure may be software implemented and may be run on any computer system having the basic components (e.g., processor, input device, user interface). The devices in various embodiments can be configured to implement all the methodologies, processes and techniques described herein. In certain implementations, the devices according to the present disclosure may include one or more processors configured to execute machine-readable instructions, a memory for storing machine-readable instructions, an input/output interface connected to the one or more processors to allow a user to interact with the device. In some embodiments the input/output interface may include a display. One or more processors may be connected to the memory to execute the machine-readable instructions comprising the steps for implementing the methodologies described herein.

For example, in some embodiments a device of the present disclosure comprises a generator configured to generate sub-microsecond long pulses of electric fields having an amplitude between 5 kV/cm and 68 kV/cm, the sub-microsecond long pulses of electric fields sufficient to suppress either one or both of myeloid-derived suppressor cell (MDSC) and regulatory T cell (Treg) production in a subject when applied to an abnormal growth of the subject; and an electrode configured to apply the sub-microsecond long pulses of electric field to the abnormal growth of the subject.

In some embodiments, the device may comprise a pre-treatment collector configured to collect pre-treatment blood of the subject prior to applying the sub-microsecond long pulses of electric fields to the abnormal growth of the subject and a post-treatment collector configured to collect post-treatment blood of the subject after applying the sub-microsecond long pulses of electric field to the abnormal growth of the subject. The device may also comprise a processor in electronic communication with computer-readable memory, the computer-readable memory storing instructions that, when executed by the processor, cause the processor to: calculate a pre-treatment measurement of a myeloid-derived suppressor cell (MDSC) concentration and/or a regulatory T cell (Treg) concentration in the pre-treatment blood; calculate a post-treatment measurement of the MDSC concentration and/or Treg concentration in the post-treatment blood; and compare the pre-treatment measurement and the post-treatment measurement. In some embodiments the results of the comparison may be displayed or otherwise indicated through a user interface.

In some embodiments, a device comprises a generator configured to generate sub-microsecond long pulses of electric fields having an amplitude between 5 kV/cm and 68 kV/cm, the sub-microsecond long pulses of electric field sufficient to stimulate release in a subject of adenosine triphosphate (ATP) and/or high mobility group box 1 (HMGB1) when applied to an abnormal growth of the subject; and an electrode configured to apply the sub-microsecond long pulses of electric field to the abnormal growth of the subject. The device may also comprise a pre-treatment collector configured to collect pre-treatment blood of the subject prior to applying the sub-microsecond long pulses of electric fields to the abnormal growth of the subject and a post-treatment collector configured to collect post-treatment blood of the subject after applying the sub-microsecond long pulses of electric field to the abnormal growth of the subject. The device may also comprise a processor in electronic communication with computer-readable memory, the computer-readable memory storing instructions that, when executed by the processor, cause the processor to: calculate a pre-treatment measurement of ATP and/or HMGB1 in the pre-treatment blood; calculate a post-treatment measurement of ATP and HMGB1 in the post-treatment blood; and compare the pre-treatment measurement and the post-treatment measurement. In some embodiments the results of the comparison may be displayed or otherwise indicated through a user interface.

In some embodiments a device comprises a generator configured to stimulate dendritic cell activation in a subject by passing sub-microsecond long pulses of electric field through an abnormal growth of the subject sufficient to increase concentration of one or more of the following: 1) a cluster of differentiation 40 (CD40), 2) a cluster of differentiation 80 (CD80), 3) a cluster of differentiation 86 (CD86), or 4) a major histocompatibility complex class II (MHC-II) molecule. The device may also comprise a processor configured to perform one or more steps of the various methods described in the present disclosure.

It will be appreciated by those skilled in the art that the invention is not limited to the use of a particular system. The subject matter of the present disclosure includes all combinations and sub-combinations of the various elements, features, functions, and/or properties disclosed herein.

What is claimed is:

1. A method for stimulating an immune response to a disease in a subject, the method comprising:
passing sub-microsecond long pulses of electric fields having an amplitude between 5 kV/cm and 68 kV/cm through an abnormal growth of a subject sufficient to suppress myeloid-derived suppressor cell (MDSC) production in the subject,
wherein at least a 35% decrease in a MDSC concentration in the subject confirms immune response stimulation.

2. The method of claim 1, further comprising:
ordering, directing or performing a comparison of a pre-treatment measurement and a post-treatment measurement of the MDSC concentration in the subject, the pre-treatment measurement occurring between 0 and 2 days before the pulses, the post-treatment measurement occurring between 2 and 7 days after the pulses.

3. The method of claim 2, further comprising:
ordering, directing or performing a calculation of a percentage change in the MDSC concentration based on the pre-treatment measurement and the post-treatment measurement.

4. The method of claim 2 further comprising:
applying a second treatment of sub-microsecond long pulses of electric fields through the abnormal growth of a subject based on the comparison.

5. The method of claim 1, further comprising:
ordering, directing, or directly performing a collection of a pre-treatment measurement, a post-treatment measurement, or both.

6. The method of claim 5 wherein the pre-treatment and post-treatment measurements are from blood samples of the subject or biopsies of the abnormal growth.

7. The method of claim 1 further comprising:
introducing an epigenetic modulator or a phosphoinositide 3-kinase (PI3K) inhibitor into a microenvironment of the abnormal growth.

8. The method of claim 7 wherein the epigenetic modulator comprises one or more of the following: 5-azacytidine, 5-aza-20-deoxycytidine, zebularine, epigallocatechin-3-gallate, suberanilohydroxamic acid (vorinostat), romidepsin, entinostat, trichostatin A (TSA), sodium butyrate, or valproic acid (VPA).

9. The method of claim 7 wherein the PI3K inhibitor comprises one or more of the following: wortmannin, demethoxyviridin, LY294002, idelalisib, perifosine, buparlisib, duvelisib, alpelisib, TGR 1202, copanlisib, PX-866, dactolisib, RP6530, SF1126, INK1117, pictilisib, XL147, XL765, palomid 529, GSK1059615, ZSTK474, PWT33597, CUDC-907, ME-401, IPI-549, IC87114, TG100-115, CAL263, RP6503, PI-103, GNE-477, or AEZS-136.

10. The method of claim 1 wherein the abnormal growth is a breast cancer tumor.

11. The method of claim 1 wherein the subject is a human.

12. The method of claim 1, the passing step comprising passing the pulses at a frequency between 0.5 Hz and 7 Hz, and each of the pulses is between 7 ns to 300 ns in duration.

13. The method of claim 1 further comprising:
suppressing production of a regulatory T cell (Treg) in the subject;
stimulating release of adenosine triphosphate (ATP) from the abnormal growth; and
stimulating a release of high mobility group box 1 (HMGB1) from the abnormal growth, wherein at least a 35% respective increase or decrease of any one or more of a Treg, ATP, or HMGB1 concentration in the subject confirms immune response stimulation.

14. A method for stimulating an immune response to a disease in a subject, the method comprising:
passing sub-microsecond long pulses of electric fields having an amplitude between 5 kV/cm and 68 kV/cm through an abnormal growth of a subject sufficient to suppress regulatory T cell (Treg) production in the subject,
wherein at least 35% decrease in a Treg concentration in the subject confirms immune response stimulation.

15. The method of claim 14, further comprising:
ordering, directing or performing a comparison of a pre-treatment measurement and a post-treatment measurement of the Treg concentration in the subject, the pre-treatment measurement occurring between 0 and 2 days before the pulses, the post-treatment measurement occurring between 2 and 7 days after the pulses.

16. The method of claim 15, further comprising:
ordering, directing or performing a calculation of a percentage change in the Treg concentration based on the pre-treatment measurement and the post-treatment measurement.

17. The method of claim 14, further comprising:
ordering, directing, or directly performing a collection of a pre-treatment measurement, a post-treatment measurement, or both.

18. The method of claim 15 wherein the pre-treatment and post-treatment measurements are from blood samples of the subject or biopsies of the abnormal growth.

19. The method of claim 14, further comprising
introducing an epigenetic modulator or a phosphoinositide 3-kinase (PI3K) inhibitor into a microenvironment of the abnormal growth.

20. The method of claim 14, the passing step comprising passing the pulses at a frequency between 0.5 Hz and 7 Hz and each of the pulses is between 7 ns to 300 ns in duration.

21. The method of claim 14, further comprising:
suppressing production of a myeloid-derived suppressor cell (MDSC) in the subject;
stimulating release of adenosine triphosphate (ATP) from the abnormal growth; and
stimulating a release of high mobility group box 1 (HMGB1) from the abnormal growth, wherein at least a 35% respective increase or decrease of any one or more of an MDSC, ATP, or HMGB1 concentration confirms immune response stimulation.

22. A device comprising:
a generator configured to generate sub-microsecond long pulses of electric fields having an amplitude between 5 kV/cm and 68 kV/cm, the sub-microsecond long pulses of electric field sufficient to suppress one or both of myeloid-derived suppressor cell (MDSC) and regulatory T cell (Treg) production in a subject when applied to an abnormal growth of the subject; and
an electrode configured to apply the sub-microsecond long pulses of electric field to the abnormal growth of the subject.

23. The device of claim 22, further comprising:
a pre-treatment collector configured to collect pre-treatment blood of the subject prior to applying the sub-microsecond long pulses of electric field to the abnormal growth of the subject;
a post-treatment collector configured to collect post-treatment blood of the subject after applying the sub-microsecond long pulses of electric field to the abnormal growth of the subject; and
a processor in electronic communication with computer-readable memory, the computer-readable memory storing instructions that, when executed by the processor, cause the processor to:
calculate a pre-treatment measurement of a myeloid-derived suppressor cell (MDSC) concentration and/or a regulatory T cell (Treg) concentration in the pre-treatment blood;
calculate a post-treatment measurement of the MDSC concentration and/or Treg concentration in the post-treatment blood; and
compare the pre-treatment measurement and the post-treatment measurement.
